(19) United States
(12) Patent Application Publication
(10) Pub. No.: US 2009/0203707 A1
(43) Pub. Date: Aug. 13, 2009

(54) METHODS FOR TREATING PAIN

(76) Inventors: Sridharan Rajamani, Fremont, CA (US); John Shryock, East Palo Alto, CA (US); Ivan Diamond, Berkeley, CA (US); Luiz Belardinelli, Palo Alto, CA (US)

Correspondence Address: CV THERAPEUTICS, INC., 3172 PORTER DRIVE, PALO ALTO, CA, 94304, US

(21) Appl. No.: 12/367,403

(22) Filed: Feb. 6, 2009

Related U.S. Application Data
(60) Provisional application No. 61/026,699, filed on Feb. 6, 2008.
(60) Provisional application No. 61/057,437, filed on May 30, 2008.

(51) Int. Cl. A61K 31/495; A61P 25/00

(52) U.S. Cl. 514/252.12

(57) ABSTRACT

This invention relates to methods for treating a patient suffering from neuropathic or nociceptive pain which may be mechanical, visceral, and/or inflammatory in nature, comprising administering a therapeutically effective amount of Ranolazine to a patient in need thereof.

This application claims priority to U.S. Provisional Patent Application Ser. No. 60/026,699, filed Feb. 6, 2008, and U.S. Provisional Patent Application Ser. No. 61/057,437, filed May 30, 2008, the entirety of which are incorporated herein by reference.

### FIELD OF THE INVENTION

This invention relates to methods for treating a patient suffering from neuropathic or nociceptive pain which may be mechanical, visceral, and/or inflammatory in nature, comprising administering a therapeutically effective amount of Ranolazine to a patient in need thereof.

### DESCRIPTION OF THE ART

U.S. Pat. No. 4,567,264, the specification of which is incorporated herein by reference in its entirety, discloses Ranolazine, (±)-N-(2,6-dimethylphenyl)-4-[2-hydroxy-3-(2-methoxyphenoxy)-propyl]-1-piperazineacetamide, and its pharmaceutically acceptable salts, and their use in the treatment of cardiovascular diseases, including arrhythmias, variant and exercise-induced angina, and myocardial infarction. In its dihydrochloride salt form, Ranolazine is represented by the formula:

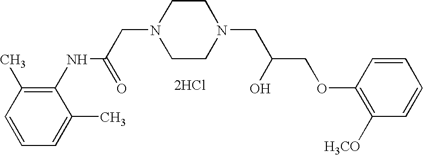

This patent also discloses intravenous (IV) formulations of dihydrochloride Ranolazine further comprising propylene glycol, polyethylene glycol 400, Tween 80 and 0.9% saline.

U.S. Pat. No. 5,506,229, which is incorporated herein by reference in its entirety, discloses the use of Ranolazine and its pharmaceutically acceptable salts and esters for the treatment of tissues experiencing a physical or chemical insult, including cardioplegia, hypoxic or reperfusion injury to cardiac or skeletal muscle or brain tissue, and for use in transplants. Oral and parenteral formulations are disclosed, including controlled release formulations. In particular, Example 7D of U.S. Pat. No. 5,506,229 describes a controlled release formulation in capsule form comprising microspheres of Ranolazine and microcrystalline cellulose coated with release controlling polymers. This patent also discloses IV Ranolazine formulations which at the low end comprise 5 mg Ranolazine per milliliter of an IV solution containing about 5% by weight dextrose. And at the high end, there is disclosed an IV solution containing 200 mg Ranolazine per milliliter of an IV solution containing about 4% by weight dextrose.

The presently preferred route of administration for Ranolazine and its pharmaceutically acceptable salts and esters is oral. A typical oral dosage form is a compressed tablet, a hard gelatin capsule filled with a powder mix or granulate, or a soft gelatin capsule (softgel) filled with a solution or suspension. U.S. Pat. No. 5,472,707, the specification of which is incorporated herein by reference in its entirety, discloses a high-dose oral formulation employing supercooled liquid Ranolazine as a fill solution for a hard gelatin capsule or softgel.

U.S. Pat. No. 6,503,911, the specification of which is incorporated herein by reference in its entirety, discloses sustained release formulations that overcome the problem of affording a satisfactory plasma level of Ranolazine while the formulation travels through both an acidic environment in the stomach and a more basic environment through the intestine, and has proven to be very effective in providing the plasma levels that are necessary for the treatment of angina and other cardiovascular diseases.

U.S. Pat. No. 6,852,724, the specification of which is incorporated herein by reference in its entirety, discloses methods of treating cardiovascular diseases, including arrhythmias variant and exercise-induced angina and myocardial infarction.

U.S. Patent Application Publication Number 2006/0177502, the specification of which is incorporated herein by reference in its entirety, discloses oral sustained release dosage forms in which the Ranolazine is present in 35-50%, preferably 40-45% Ranolazine. In one embodiment the Ranolazine sustained release formulations of the invention include a pH dependent binder; a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example, sodium hydroxide, potassium hydroxide, or ammonium hydroxide, in a quantity sufficient to neutralize the methacrylic acid copolymer to an extent of about 1-20%, for example about 3-6%. Suitable pH independent binders include, but are not limited to, hydroxypropylmethylcellulose (HPMC), for example Methocel® E10M Premium CR grade HPMC or Methocel® E4M Premium HPMC. Suitable pharmaceutically acceptable excipients include magnesium stearate and microcrystalline cellulose (Avicel® pH101).

### BACKGROUND

Physical pain may be defined in a number of ways but generally falls within two classifications, nociceptive and neuropathic. Nociceptive pain is pain that is triggered by stimulation of sensory receptive nerve endings called nociceptors which are located through out the body in the various tissues such as skin, cornea, mucosa, muscle, and joint. The essential functions of nociceptors include the transduction of noxious stimuli into depolarizations that trigger action potentials, conduction of action potentials from primary sensory sites to synapses in the central nervous system, and conversion of action potentials into neurotransmitter release at presynaptic terminals. Nociceptive pain is typically experienced as a consequence of sprains, bone fractures, burns, bumps, bruises, and inflammation (from an infection or arthritic disorder), i.e. any damage to tissues that leads to activation of nociceptors.

The number and type of nociceptors is highly dependent upon their location within the body. Cutaneous nociceptors located in the skin are highly concentrated and result in well-defined localized pain. Somatic nociceptors in the body's ligaments, connective tissues, and bones are much less numerous resulting in poorly-localized, aching pain which may be experienced for a longer duration. Even less numerous are visceral nociceptors located in the body's organs and viscera. Consequently, the source of visceral pain is often extremely difficult to identify.

In contrast, neuropathic pain is pain that is initiated or caused by a primary lesion or dysfunction of the nervous system itself. Neuropathic pain is usually perceived as a steady burning and/or “pins and needles” and/or “electric shock” sensations and/or tickling. The difference is due to the fact that “ordinary” pain stimulates only pain nerves, while a neuropathy often results in the firing of both pain and non-pain (touch, warm, cool) sensory nerves in the same area, producing signals that the spinal cord and brain do not normally expect to receive. Neuropathic pain may also be caused by over activity of nociceptors themselves. This over activity may be the result of an increase or decrease in the numbers, locations, or functions of cell membrane ion channels themselves.

The four major types of nerve damage are polyneuropathy, autonomic neuropathy, mononeuropathy, and mononeuritis multiplex. This type of pain has many manifestations and causes and can be acute or chronic (persistent), with the latter type most often seen in clinical practice. By one estimate, neuropathic pain affects at least 1.5% of the US population with neuropathic back and leg pain and diabetic neuropathy having the highest prevalence. Between 8-50% of diabetics are estimated to have symptoms of diabetic neuropathy, and 10-19% of back pain patients are estimated to have neuropathic pain. (See, Taylor R S (2006) Pain Practice; 6: 22-26)

For a number of reasons the true prevalence of neuropathic pain is difficult to ascertain. For example it is not clear how many instances of common low back pain are neuropathic in origin. The difficulty is compounded by the fact that neuropathic pain is often a symptom or consequence of another underlying chronic disease. Typically, the physician's emphasis is on the diagnosis and treatment of the primary disease often resulting in neuropathic pain being under-diagnosed and under-treated.

Current treatments for pain include analgesics such as acetaminophen and anti-inflammatory drugs including glucocoricoidsteroids like hydrocortisone, prednisone, and dexamethazone, and non-steroidal anti-inflammatory drugs (NSAIDs) like ibuprofen, aspirin, naproxen, and celecoxib (Celebrex). Stronger medications include opioids morphine, codeine, oxycodone, heroin, fentanyl, and hydrocone. Other treatments for neuropathic pain include tricyclic antidepressants such as amitriptyline (Elavil®), anticonvulsants like valproate, carbamazepine (Tegretol®), and capsaicin.

Unfortunately each of the aforementioned drug classes posses several drawbacks which limit their utility and effectiveness. Analgesics have limited potency. Glucocoricoidsteroids cause changes to the immune system, delay wound healing, inhibit bone formation, and suppress calcium absorption while NSAIDs have gastrointestinal side effects as well as other concerns regarding cardiovascular effects. Opioids are notoriously addictive and have other side effects such as nausea, vomiting, respiratory depression, and constipation. Tricyclic antidepressants and anticonvulsants also have significant drawbacks. Clearly there is a need for safer and more efficacious medications.

It has now been discovered that at therapeutic drug concentrations Ranolazine blocks both the Na_{V}1.7 and Nav1.8 sodium current (I_{Na}) in HEK293 cells stably expressing hNa_{V}1.7 and ND-7-23 cells stably expressing rNav1.8. The preliminary finding that Ranolazine is a relatively selective blocker of peak and window Na_{V}1.7 and Nav1.8 currents, and the demonstrated safety of the drug in humans, give strong support to the use of Ranolazine for treatment of nociceptive pain and neuropathic pain.

### SUMMARY OF THE INVENTION

The object of the invention is to provide methods for the treatment or prevention of pain comprising the step of administering to a patient in need thereof a therapeutically effective amount, or a prophylactically effective amount, of Ranolazine, or a pharmaceutically acceptable salt thereof.

In some aspects of the invention, Ranolazine is administered for the treatment or prevention of neuropathic or nociceptive pain. When nociceptive pain is to be treated it may be mechanical, chemical, and/or inflammatory in nature. When Ranolazine is administered for the treatment or prevention of neuropathic pain, the pain may be associated with a sodium channelopathy, polyneuropathy, autonomic neuropathy, mononeuropathy, and/or mononeuritis multiplex. Treatable channelopathies include, but are not limited to, erythromelalgia and paroxysmal extreme pain disorder. Treatable conditions associated with sodium channelopathies include, but are not limited to, myotonia and muscle paralysis.

In other aspects of the invention the pain may be the result of chronic, visceral, mechanical, inflammatory and/or neuropathic pain syndromes. The pain may also be resulting from, or associated with, traumatic nerve injury, nerve compression or entrapment, postherpetic neuralgia, trigeminal neuralgia, diabetic neuropathy, cancer and chemotherapy. Additional indications for with the method of the invention is suitable include, but are not limited to, chronic lower back pain, HIV- and HIV treatment-induced neuropathy, cancer treatment-induced, i.e., chemotherapy-induced neuropathy, chronic pelvic pain, neuroma pain, complex regional pain syndrome, chronic arthritic pain, and related neuralgias.

### DESCRIPTION OF THE DRAWINGS

FIG. 1 depicts the concentration-dependent block of Na_{V}1.7 peak I_{Na} by Ranolazine, R-Ranolazine and S-Ranolazine as described in Example 1. Data were fit with Hill equation.

FIG. 2 presents a plot of peak I_{Na} values with repetitive pulses with duration of 2, 5, 20, and 200 msec normalized to a value recorded in response to first depolarizing step in the absence (open symbols) and presence of 100 μM Ranolazine (filled symbols) as described in Example 1. The block reaches the same level with pulse duration as short as 2 msec.

FIGS. 3A, 3B and 3C illustrates the effect of 300 nM TTX to reduce I_{Na} in HEK293 cells stably expressing hNav1.7+β1 subunits (A) and in untransfected ND7-23 cells (B) or ND7-23/rNav1.8 Na⁺ channels (C), as discussed in Example 2. Whole-cell currents were recorded during a 50-msec test pulse to −20 (hNav1.7 or untransfected ND7-23 cells) or +20 (rNav1.8) mV at intervals of 10 sec. Addition of 300 nM TTX completely blocked the hNav1.7 I_{Na} (A). However, 300 nM TTX caused minimal block of rNav1.8 I_{Na} (C), demonstrating and confirming the reported resistance of this channel isoform to TTX.

FIG. 4A presents representative records of I_{Na} recorded in the absence and presence of 30 μM ranolazine from HEK293 cells stably expressing hNav1.7+β1 subunits and from ND7-23 cells stably expressing rNav1.8 Na⁺ channels as discussed in Example 2. Whole-cell currents were recorded during a 50-msec test pulse from −120 to −20 (hNav1.7) or −100 mV to +20 (rNav1.8) mV in intervals of 10 sec. FIG. 4B shows the concentration-response relationships for ranolazine to reduce I_{Na} of hNav1.7 (▪, n=4-6 cells, each) and rNav1.8 (, n=4-6 cells, each) Na⁺ channels. Data represent mean±SEM. Sensitivity to ranolazine of hNav1.7 or rNav1.8 Na⁺ channels in the inactivated state was determined using a 5-sec prepulse to −70 mV for hNav1.7 (□, n=4 cells, each) or −40 mV for rNav1.8 (∘, n=3-5 cells, each) followed by a 20-msec step to the holding potential (−120 or −100 mV) before a 50-msec depolarizing step to −20 or +20 mV. The 20-msec step was chosen to be short, to allow channel recovery from inactivation with minimal drug dissociation from blocked channels.

FIG. 5 shows the current-voltage relationships for the effects of ranolazine on activation and inactivation of hNav1.7 and rNav1.8 Na⁺ channel currents as discussed in Example 2. FIG. 5A representative I_{Na} records from HEK293 cells expressing hNav1.7+β1 subunits, and from ND7-23 cells expressing rNav1.8 Na⁺ channels. FIG. 5B presents activation curves for hNav1.7+β1 subunits and rNav1.8 Na⁺ channels in the absence (▪, ) and presence (□, ∘) of 10 μM ranolazine. The smooth curves are Boltzmann fits with mid-points (V_{1/2}) and slope factors (k) given in Table 5. FIG. 5C shows inactivation time constants of hNav1.7 (left panel) and rNav1.8 (right panel) I_{Na} plotted versus voltage (currents described in FIG. 3B) in the absence and presence of 10 μM ranolazine fit to a single exponential equation. Data represent mean±SEM.

FIGS. 6A-C depicts voltage dependence of steady-state inactivation for hNav1.7 (left panels) and rNav1.8 (right panels) Na⁺ channel currents in the absence (filled symbols) and presence of 10 μM ranolazine (open symbols) as discussed in Example 3. Conditioning prepulses of 100 msec (FIG. 6A), 1 sec (FIG. 6B) and 10 sec (FIG. 6C) were used. Inset: voltage-clamp protocols. FIG. 6A: ranolazine 10 μM caused a minimal shift in the mid-point (V_{1/2}) without affecting the slope factor (k) of steady-state fast inactivation of hNav1.7 (n=4 cells) and rNav1.8 (n=4 cells). The estimated V_{1/2} and k values in the absence (▪) and presence of ranolazine (□) for hNav1.7 are −74.49±2.79; 6.01±0.3 and −86.15±3.62 (p<0.05); 7.55±0.82 (p=0.14), and the estimated V_{1/2} and k values in the absence () and presence of ranolazine (∘) for rNav1.8 are −33.12±1.10; 9.69±1.10 and −40.66±3.23 (p=0.15); 11.45±1.21 (p<0.02), respectively. FIG. 6B: ranolazine caused a concentration-dependent (1-30 μM) shift in the V_{1/2} of steady-state intermediate inactivation without affecting k values for both hNav1.7 and rNav1.8 (Table 5). FIG. 6C: ranolazine (10 μM) caused a significant leftward shift in the V_{1/2} of steady-state slow inactivation without affecting the k values of hNav1.7 (n=4 cells) and rNav1.8 (n=6 cells). The estimated V_{1/2} and k values in the absence (▪) and presence of ranolazine (□) for hNav1.7 are −37.22±4.21; 13.52±0.93 and −61.39±3.54 (p<0.05); 14.22±2.14 p=0.80) and the estimated V_{1/2} and k values in the absence () and presence of ranolazine (∘) for rNav1.8 are −37.13±2.42; 7.31±0.81 and −54.57±3.69 (p<0.05); 8.38±0.76 (p=0.23), respectively. Data represent mean±SEM.

FIG. 7 plots the development of slow inactivation in the absence and presence of 30 μM ranolazine (inset: voltage-clamp protocol). Data represent mean±SEM. The smooth curves are fits of the data with two (FIG. 7A; h Nav1.7, n=3-5 cells, each) or three (FIG. 7B; rNav1.8, n=3-5 cells, each) component exponential equations (see Table 6 for values of the individual parameters). FIG. 7C and FIG. 7D are plots of recovery from inactivation in the absence and presence of 30 μM ranolazine (inset: voltage-clamp protocol). Data represent mean±SEM. The smooth curves are fits of the data with two (FIG. 7C; h Nav1.7, n=5 cells, each) or three (FIG. 7D; rNav1.8, n=5 cells, each) component exponential equations (see Table 6 for values of the individual parameters).

FIG. 8 plots use-dependent block of hNav1.7 (FIG. 8A), rNav1.8 (FIG. 8B) and TTX-S I_{Na} (C) by 30 μM ranolazine as discussed in Example 2. Each protocol included a train of 40 pulses from −120 to −20 mV (Nav1.7+β1 or endogenous TTX-S I_{Na}) or from −100 to +50 mV (rNav1.8) at frequencies of 1, 5 and 10 Hz in the absence (control; filled symbols) or presence of 30 μM ranolazine (open symbols). The amplitude of currents evoked by the n^{th} impulse (40^{th}) was normalized to that of the current evoked by the first pulse and plotted versus respective pulse number.

FIG. 9 shows the effect of increased pulse duration on the use dependence of ranolazine block of rNav1.8 I_{Na} as discussed in Example 2. Consecutively-recorded rNav1.8 I_{Na} traces in the presence of 100 μM ranolazine. A total of 40 pulses (p) to +50 mV with durations of either 5 (FIG. 9A) or 200 ms (FIG. 9B) were applied at a frequency of 5 Hz; the pulse number is indicated. FIG. 9C presents plots of rNav1.8 I_{Na} measured at +50 mV using pulses of 3 (∇), 5 (∇), 20 (∘) or 200 (□) msec duration in the presence of 100 μM ranolazine. Current amplitude elicited by each pulse was normalized to the peak amplitude of current elicited by the first pulse (1P).

FIG. 10 depicts the results of ranolazine treatment of CFA-induced thermal and mechanical hyperalgesia following intraperitoneal administration as discussed in Example 3. FIG. 10A depicts no significant effect of treatment on paw withdrawal from thermal stimulation. By contrast, FIG. 10B depicts a dose dependent reduction in mechanical allodynia.

FIG. 11 depicts the results of ranolazine treatment of CFA-induced thermal and mechanical hyperalgesia following oral administration. As in FIG. 10A, FIG. 11A depicts no significant effect of treatment on paw withdrawal from thermal stimulation. FIG. 11B, however, depicts a dose dependent reduction in mechanical allodynia. Optimum oral dosing was achieved at 50 mg/kg. No additional benefit was observed at higher doses.

### DETAILED DESCRIPTION OF THE INVENTION

### Definitions

In this specification and in the claims that follow, reference will be made to a number of terms that shall be defined to have the following meanings.

“Ranolazine”, when referred to as Ranexa®, is the compound (±)-N-(2,6-dimethylphenyl)-4-[2-hydroxy-3-(2-methoxyphenoxy)propyl]-1-piperazine-acetamide. Ranolazine can also exist as its enantiomers (R)-(+)-N-(2,6-dimethylphenyl)-4-[2-hydroxy-3-(2-methoxyphenoxy)-propyl]-1-piperazineacetamide (also referred to as R-Ranolazine), and (S)-(−)-N-(2,6-dimethylphenyl)-4-[2-hydroxy-3-(2-methoxyphenoxy)-propyl]-1-piperazineacetamide (also referred to as S-Ranolazine), and their pharmaceutically acceptable salts, and mixtures thereof. Unless otherwise stated the Ranolazine plasma concentrations used in the specification and examples refer to Ranolazine free base. At pH ˜4, in an aqueous solution titrated with hydrogen chloride, Ranolazine will be present in large part as its dihydrochloride salt.

“Physiologically acceptable pH” refers to the pH of an intravenous solution which is compatible for delivery into a human patient. Preferably, physiologically acceptable pH's range from about 4 to about 8.5 and preferably from about 4 to 7. Without being limited by any theory, the use of intravenous solutions having a pH of about 4 to 6 are deemed physiologically acceptable as the large volume of blood in the body effectively buffers these intravenous solutions.

“Cardiovascular diseases” or “cardiovascular symptoms” refer to diseases or symptoms exhibited by, for example, heart failure, including congestive heart failure, acute heart failure, ischemia, recurrent ischemia, myocardial infarction, STEMI and NSTEMI, and the like, arrhythmias, angina, including exercise-induced angina, variant angina, stable angina, unstable angina, acute coronary syndrome, NSTEACS, and the like, diabetes, and intermittent claudication. The treatment of such disease states is disclosed in various U.S. patents and patent applications, including U.S. Pat. Nos. 6,503,911 and 6,528,511, U.S. Patent Application Nos. 2003/0220344 and 2004/0063717, the complete disclosures of which are hereby incorporated by reference.

“Inhibitor” refers to a compound that “slows down” the metabolism of a substrate. Inhibitors may be classified into strong, moderate and weak categories. Strong inhibitors, for example including bupropion, fluoxetine, paroxetine, and quinidine, can cause a >5-fold increase in the plasma AUC values or more than 80% decrease in clearance. Moderate inhibitors, for example including duloxetine and terbinafine, can cause a >2-fold increase in the plasma AUC values or 50-80% decrease in clearance. Weak inhibitors, for example including amiodarone and cimetidine, can cause a >1.25-fold but <2-fold increase in the plasma AUC values or 20-50% decrease in clearance.

““Optional” and “optionally” mean that the subsequently described event or circumstance may or may not occur, and that the description includes instances where the event or circumstance occurs and instances in which it does not. For example, “optional pharmaceutical excipients” indicates that a formulation so described may or may not include pharmaceutical excipients other than those specifically stated to be present, and that the formulation so described includes instances in which the optional excipients are present and instances in which they are not.

“Treating” and “treatment” refer to any treatment of a disease in a patient and include: preventing the disease from occurring in a subject which may be predisposed to the disease but has not yet been diagnosed as having it; inhibiting the disease, i.e., arresting its further development; inhibiting the symptoms of the disease; relieving the disease, i.e., causing regression of the disease, or relieving the symptoms of the disease. The “patient” is a mammal, preferably a human.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, and the age, physical condition, existence of other disease states, and nutritional status of the patient. Additionally, other medication the patient may be receiving will effect the determination of the therapeutically effective amount of the therapeutic agent to administer.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Ranolazine, which is named N-(2,6-dimethylphenyl)-4-[2-hydroxy-3-(2-methoxyphenoxy)propyl]-1-piperazineacetamide {also known as 1-[3-(2-methoxyphenoxy)-2-hydroxypropyl]-4-[(2,6-dimethylphenyl)-aminocarbonylmethyl]-piperazine}, can be present as a racemic mixture, or an enantiomer thereof, or a mixture of enantiomers thereof, or a pharmaceutically acceptable salt thereof. Ranolazine can be prepared as described in U.S. Pat. No. 4,567,264, the specification of which is incorporated herein by reference.

“Immediate release” (“IR”) refers to formulations or dosage units that rapidly dissolve in vitro and are intended to be completely dissolved and absorbed in the stomach or upper gastrointestinal tract. Conventionally, such formulations release at least 90% of the active ingredient within 30 minutes of administration.

“Sustained release” (“SR”) refers to formulations or dosage units used herein that are slowly and continuously dissolved and absorbed in the stomach and gastrointestinal tract over a period of about six hours or more. Preferred sustained release formulations are those exhibiting plasma concentrations of Ranolazine suitable for no more than twice daily administration with two or less tablets per dosing as described below.

“Isomers” are different compounds that have the same molecular formula.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other.

The absolute stereochemistry is specified according to the Cahn-Ingold-Prelog R—S system. When the compound is a pure enantiomer the stereochemistry at each chiral carbon may be specified by either R or S. Resolved compounds whose absolute configuration is unknown are designated (+) or (−) depending on the direction (dextro- or laevorotary) which they rotate the plane of polarized light at the wavelength of the sodium D line.

“Polyneuropathy” is defined as a neurological disorder occurring when many peripheral nerves throughout the body malfunction simultaneously. It may be acute or chronic.

“Autonomic neuropathy” as used herein refers to a group of symptoms caused by damage to nerves that regulate blood pressure, heart rate, bowel and bladder emptying, digestion, and other body functions.

“Mononeuropathy” is defined as a type of neuropathy affecting only a single peripheral or cranial nerve. Common type of mononeuropathies include, but are not limited to, thoracic outlet syndrome, carpal tunnel syndrome, radial neuropathy, winged scapula, meralgia paraesthetica, tarsal tunnel syndrome, oculomotor nerve palsy, fourth nerve palsy, sixth nerve palsy, and Bell's palsy.

“Mononeuritis multiplex” is defined a neurological disorder that involves damage to at least two separate nerve areas. It is a form of peripheral neuropathy (damage to nerves outside the brain and spinal cord). Common causes include a lack of oxygen caused by decreased blood flow or inflammation of blood vessels. No cause is identified for about a third of cases. Other common causes of mononeuritis multiplex include, but are not limited to, Diabetes mellitus, blood vessel diseases such as polyarteritis nodosa, and connective diseases such as rheumatoid arthritis or systemic lupus erythematosus.

“Channelopathy” refers to a disease or condition that is associated with ion channel malformation. Examples of sodium channelopathies include, but are not limited to erythromelalgia and paroxysmal extreme pain disorder.

Ranolazine is capable of forming acid and/or base salts by virtue of the presence of amino and/or carboxyl groups or groups similar thereto. The term “pharmaceutically acceptable salt” refers to salts that retain the biological effectiveness and properties of Ranolazine and which are not biologically or otherwise undesirable. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl)amines, tri(substituted alkyl)amines, alkenyl amines, dialkenyl amines, trialkenyl amines, substituted alkenyl amines, di(substituted alkenyl)amines, tri(substituted alkenyl)amines, cycloalkyl amines, di(cycloalkyl)amines, tri(cycloalkyl)amines, substituted cycloalkyl amines, disubstituted cycloalkyl amine, trisubstituted cycloalkyl amines, cycloalkenyl amines, di(cycloalkenyl)amines, tri(cycloalkenyl)amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl)amine, tri(n-propyl)amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### Methods of the Invention

The method of the invention is based on the surprising discovery that Ranolazine blocks both the Na_{V}1.7 and Nav1.8 currents at therapeutic drug concentrations. Ranolazine inhibits both peak and “window” Na_{V}1.7 and Nav1.8 currents. On the other hand, Ranolazine selectively inhibits late relative to peak Na_{V}1.5 current, and does not appear to block Na_{V}1.1, 1.4 or 1.6 peak currents at therapeutic concentrations. The finding that Ranolazine is a relatively selective blocker of peak and window Na_{V}1.7 and Nav1.8 currents, and the demonstrated safety of the drug in humans, give strong support to the use of Ranolazine for treatment of nociceptive pain and the treatment of neuropathic pain that is caused by inherited or acquired sodium channelopathies.

Pathophysiological mechanisms of neuropathic pain have been proposed from experimental work in animal models and from an elucidation of hereditary causes of altered sensitivity to painful stimuli. Studies of small dorsal root ganglia (DRG) cells, which are primary afferent nociceptors that project to the dorsal horn of the spinal cord, have been particularly informative. Spinal dorsal root ganglia are not protected by a blood-brain barrier and may be accessible to systemic drug therapy. These cells express several isoforms of the alpha (pore-forming) subunit of the voltage-gated sodium channel (e.g., Na_{V}1.3, 1.6, 1.7, 1.8, 1.9).

These various Na⁺ channel isoforms are known to have different properties and roles in DRG function. The Na⁺ channel isoform Na_{V}1.7 is highly expressed in DRG neurons and expression is further increased in DRG neurons from rats rendered diabetic by administration of streptozotocin. (See, Hong et al. (2004) J Biol Chem 279: 29341-29350) Increased expression of Na_{V}1.7 in rat DRG neurons correlated with increased Na⁺ current density and with the development of hyperalgesia (an increased response to a stimulus that is normally painful) and allodynia (pain elicited by a stimulus that does not normally provoke pain). (Hong et al. (2004))

Evidence from studies of humans also implicates Na_{V}1.7 in pain perception. Congenital insensitivity to pain is present in persons with nonsense “loss-of-function” mutations in the gene encoding Na_{V}1.7, (See, Cox et al (2006) Nature 444: 894-898) and chronic pain and hyperalgesia is present in persons with “gain-of-function” missense mutations in Na_{V}1.7, such as those causing erythromelalgia, Cummings et al, (2007) Pain 131:243-257. These findings suggest that a mechanistic approach to treatment of neuropathic pain using drugs that alter the function of specific isoforms of Na⁺ channels (e.g., Na_{V}1.7) is a rational therapeutic plan.

Nav1.8 is a slowly-inactivating TTX-R Na⁺ channel that is found in DRG cells and small nociceptive C-type pain fibers (Akopian et al, 1996; Sangameswaran et al., 1996). The gene SCN10A encodes the alpha polypeptide of Nav1.8 (Akopian et al., 1996; Sangameswaran et al., 1996).

There is a great need for new drugs to treat pain (Markman and Dworkin, 2006; Flugsrud-Dreckenridge et al., 2007). Because evidence from many studies suggests that both Nav1.7 and Nav1.8 play critical roles in peripheral pain sensing, blocking both or either one of these Na⁺ channel isoforms is a potentially important treatment to alleviate pain. The Na⁺ channel blockers lidocaine (a local anesthetic) and mexiletine (a lidocaine analogue) have been shown to attenuate hyperalgesia in animal models of neuropathic pain and in humans (Jarvis and Coukell, 1998; Jett et al., 1997). Recently, it was reported that ranolazine blocked neuronal Nav1.7 Na⁺ current (I_{Na}) in a state and use-dependent manner (Wang et al, 2008). Ranolazine reduces the persistent (late) Na⁺ current (late I_{Na}) in the heart (Belardinelli et al., 2006), and the drug has been approved for reduction of chronic angina, and shown to be safe (Scirica et al, 2007).

Several classes of drugs that act as sodium channel blockers are used to treat neuropathic pain. These include local anesthetic (e.g., lidocaine), anti-arrhythmic (e.g., mexiletine), and anti-epileptic (e.g., phenyloin, carbamazepine) drugs. None of these drugs is a selective blocker of Na_{V}1.7 or of any other Na⁺ channel subtype. They may act to stabilize inactivated states of Na⁺ channels and cause use-dependent block of channel activity, thereby reducing the maximum rate of neuronal firing. Their reported efficacy is only partial, Drenth et al. (2007) J Clin Invest, 117:3603-3609, and their use is associated with CNS (e.g., tremor, seizures) or cardiac (arrhythmias) toxicity. Na⁺ channel subtype-selective blockers are a current focus of therapeutics.

### Utility Testing and Administration

### General Utility

The method of the invention is useful for treating pain arising from a wide variety of causes. While not wishing to be bound by theory, it is believe that the ability of Ranolazine to treat pain stems is a result of its surprising capacity to act as a selective blocker of peak and window Nav1.7 and Nav1.8 currents.

### Pharmaceutical Compositions and Administration

Ranolazine is usually administered in the form of a pharmaceutical composition. This invention therefore provides pharmaceutical compositions that contain, as the active ingredient, Ranolazine, or a pharmaceutically acceptable salt or ester thereof, and one or more pharmaceutically acceptable excipients, carriers, including inert solid diluents and fillers, diluents, including sterile aqueous solution and various organic solvents, solubilizers and adjuvants. Ranolazine may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3^{rd} Ed. (G. S. Banker & C. T. Rhodes, Eds.).

The Ranolazine may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

Oral administration is the preferred route for administration of Ranolazine. Administration may be via capsule or enteric coated tablets, or the like. In making the pharmaceutical compositions that include Ranolazine, the active ingredient is usually diluted by an excipient and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 50% by weight of the active compound, soft and hard gelatin capsules, sterile injectable solutions, and sterile packaged powders.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Ranolazine is effective over a wide dosage range and is generally administered in a pharmaceutically effective amount. Typically, for oral administration, each dosage unit contains from 1 mg to 2 g of Ranolazine, more commonly from 1 to 700 mg, and for parenteral administration, from 1 to 700 mg of Ranolazine, more commonly about 2 to 200 mg. It will be understood, however, that the amount of Ranolazine actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

One mode for administration is parental, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the invention in the required amount in the appropriate solvent with various other ingredients as enumerated above, as required, followed by filtration and sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The intravenous formulation of Ranolazine is manufactured via an aseptic fill process as follows. In a suitable vessel, the required amount of Dextrose Monohydrate is dissolved in Water for Injection (WFI) at approximately 78% of the final batch weight. With continuous stirring, the required amount of Ranolazine free base is added to the dextrose solution. To facilitate the dissolution of Ranolazine, the solution pH is adjusted to a target of 3.88-3.92 with 0.1N or 1N Hydrochloric Acid solution. Additionally, 0.1N HCl or 1.0N NaOH may be utilized to make the final adjustment of solution to the target pH of 3.88-3.92. After Ranolazine is dissolved, the batch is adjusted to the final weight with WFI. Upon confirmation that the in-process specifications have been met, the Ranolazine bulk solution is sterilized by sterile filtration through two 0.2 μm sterile filters. Subsequently, the sterile Ranolazine bulk solution is aseptically filled into sterile glass vials and aseptically stoppered with sterile stoppers. The stoppered vials are then sealed with clean flip-top aluminum seals.

Ranolazine may be impregnated into a stent by diffusion, for example, or coated onto the stent such as in a gel form, for example, using procedures known to one of skill in the art in light of the present disclosure.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). Ranolazine is effective over a wide dosage range and are generally administered in a pharmaceutically effective amount. Preferably, for oral administration, each dosage unit contains from 10 mg to 2 g of a compound Ranolazine, more preferably 10 to 1500 mg, more preferably from 10 to 1000 mg, more preferably from 500 to 1000 mg. It will be understood, however, that the amount of Ranolazine actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

In one embodiment, the Ranolazine is formulated so as to provide quick, sustained or delayed release of the active ingredient after administration to the patient, especially sustained release formulations. Unless otherwise stated, the Ranolazine plasma concentrations used in the specification and examples refer to Ranolazine free base.

The preferred sustained release formulations of this invention are preferably in the form of a compressed tablet comprising an intimate mixture of compound and a partially neutralized pH-dependent binder that controls the rate of dissolution in aqueous media across the range of pH in the stomach (typically approximately 2) and in the intestine (typically approximately about 5.5). An example of a sustained release formulation is disclosed in U.S. Pat. Nos. 6,303,607; 6,479,496; 6,369,062; and 6,525,057, the complete disclosures of which are hereby incorporated by reference.

To provide for a sustained release of Ranolazine, one or more pH-dependent binders are chosen to control the dissolution profile of the compound so that the formulation releases the drug slowly and continuously as the formulation passed through the stomach and gastrointestinal tract. The dissolution control capacity of the pH-dependent binder(s) is particularly important in a sustained release formulation because a sustained release formulation that contains sufficient compound for twice daily administration may cause untoward side effects if the compound is released too rapidly (“dose-dumping”).

Accordingly, the pH-dependent binders suitable for use in this invention are those which inhibit rapid release of drug from a tablet during its residence in the stomach (where the pH is below about 4.5), and which promotes the release of a therapeutic amount of compound from the dosage form in the lower gastrointestinal tract (where the pH is generally greater than about 4.5). Many materials known in the pharmaceutical art as “enteric” binders and coating agents have the desired pH dissolution properties. These include phthalic acid derivatives such as the phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof.

Preferred pH-dependent binder materials that can be used in conjunction with the compound to create a sustained release formulation are methacrylic acid copolymers. Methacrylic acid copolymers are copolymers of methacrylic acid with neutral acrylate or methacrylate esters such as ethyl acrylate or methyl methacrylate. A most preferred copolymer is methacrylic acid copolymer, Type C, USP (which is a copolymer of methacrylic acid and ethyl acrylate having between 46.0% and 50.6% methacrylic acid units). Such a copolymer is commercially available, from Röhm Pharma as Eudragit® L 100-55 (as a powder) or L30D-55 (as a 30% dispersion in water). Other pH-dependent binder materials which may be used alone or in combination in a sustained release formulation dosage form include hydroxypropyl cellulose phthalate, hydroxypropyl methylcellulose phthalate, cellulose acetate phthalate, polyvinylacetate phthalate, polyvinylpyrrolidone phthalate, and the like.

One or more pH-independent binders may be in used in sustained release formulations in oral dosage forms. It is to be noted that pH-dependent binders and viscosity enhancing agents such as hydroxypropyl methylcellulose, hydroxypropyl cellulose, methylcellulose, polyvinylpyrrolidone, neutral poly(meth)acrylate esters, and the like, may not themselves provide the required dissolution control provided by the identified pH-dependent binders. The pH-independent binders may be present in the formulation of this invention in an amount ranging from about 1 to about 10 wt %, and preferably in amount ranging from about 1 to about 3 wt % and most preferably about 2.0 wt %.

As shown in Table 1, Ranolazine, is relatively insoluble in aqueous solutions having a pH above about 6.5, while the solubility begins to increase dramatically below about pH 6.

TABLE 1

| Solution pH | Solubility (mg/mL) | USP Solubility Class |
|---|---|---|
| 4.81 | 161 | Freely Soluble |
| 4.89 | 73.8 | Soluble |
| 4.90 | 76.4 | Soluble |
| 5.04 | 49.4 | Soluble |
| 5.35 | 16.7 | Sparingly Soluble |
| 5.82 | 5.48 | Slightly soluble |
| 6.46 | 1.63 | Slightly soluble |
| 6.73 | 0.83 | Very slightly soluble |
| 7.08 | 0.39 | Very slightly soluble |
| 7.59 (unbuffered water) | 0.24 | Very slightly soluble |
| 7.79 | 0.17 | Very slightly soluble |
| 12.66 | 0.18 | Very slightly soluble |

Increasing the pH-dependent binder content in the formulation decreases the release rate of the sustained release form of the compound from the formulation at pH is below 4.5 typical of the pH found in the stomach. The enteric coating formed by the binder is less soluble and increases the relative release rate above pH 4.5, where the solubility of compound is lower. A proper selection of the pH-dependent binder allows for a quicker release rate of the compound from the formulation above pH 4.5, while greatly affecting the release rate at low pH. Partial neutralization of the binder facilitates the conversion of the binder into a latex like film which forms around the individual granules. Accordingly, the type and the quantity of the pH-dependent binder and amount of the partial neutralization composition are chosen to closely control the rate of dissolution of compound from the formulation.

The dosage forms of this invention should have a quantity of pH-dependent binders sufficient to produce a sustained release formulation from which the release rate of the compound is controlled such that at low pHs (below about 4.5) the rate of dissolution is significantly slowed. In the case of methacrylic acid copolymer, type C, USP (Eudragit® L 100-55), a suitable quantity of pH-dependent binder is between 5% and 15%. The pH dependent binder will typically have from about 1 to about 20% of the binder methacrylic acid carboxyl groups neutralized. However, it is preferred that the degree of neutralization ranges from about 3 to 6%. The sustained release formulation may also contain pharmaceutical excipients intimately admixed with the compound and the pH-dependent binder. Pharmaceutically acceptable excipients may include, for example, pH-independent binders or film-forming agents such as hydroxypropyl methylcellulose, hydroxypropyl cellulose, methylcellulose, polyvinylpyrrolidone, neutral poly(meth)acrylate esters (e.g. the methyl methacrylate/ethyl acrylate copolymers sold under the trademark Eudragit® NE by Röhm Pharma, starch, gelatin, sugars carboxymethylcellulose, and the like. Other useful pharmaceutical excipients include diluents such as lactose, mannitol, dry starch, microcrystalline cellulose and the like; surface active agents such as polyoxyethylene sorbitan esters, sorbitan esters and the like; and coloring agents and flavoring agents. Lubricants (such as tale and magnesium stearate) and other tableting aids are also optionally present.

The sustained release formulations of this invention have an active compound content of above about 50% by weight to about 95% or more by weight, more preferably between about 70% to about 90% by weight and most preferably from about 70 to about 80% by weight; a pH-dependent binder content of between 5% and 40%, preferably between 5% and 25%, and more preferably between 5% and 15%; with the remainder of the dosage form comprising pH-independent binders, fillers, and other optional excipients.

One particularly preferred sustained release formulations of this invention is shown below in Table 2.

TABLE 2

| | | Preferred | |
|---|---|---|---|
| | Weight Range | Range | Most |
| Ingredient | (%) | (%) | Preferred |
| Active ingredient | 50-95 | 70-90 | 75 |
| Microcrystalline cellulose (filler) | 1-35 | 5-15 | 10.6 |
| Methacrylic acid copolymer | 1-35 | 5-12.5 | 10.0 |
| Sodium hydroxide | 0.1-1.0 | 0.2-0.6 | 0.4 |
| Hydroxypropyl methylcellulose | 0.5-5.0 | 1-3 | 2.0 |
| Magnesium stearate | 0.5-5.0 | 1-3 | 2.0 |

The sustained release formulations of this invention are prepared as follows: compound and pH-dependent binder and any optional excipients are intimately mixed (dry-blended). The dry-blended mixture is then granulated in the presence of an aqueous solution of a strong base that is sprayed into the blended powder. The granulate is dried, screened, mixed with optional lubricants (such as talc or magnesium stearate), and compressed into tablets. Preferred aqueous solutions of strong bases are solutions of alkali metal hydroxides, such as sodium or potassium hydroxide, preferably sodium hydroxide, in water (optionally containing up to 25% of water-miscible solvents such as lower alcohols).

The resulting tablets may be coated with an optional film-forming agent, for identification, taste-masking purposes and to improve ease of swallowing. The film forming agent will typically be present in an amount ranging from between 2% and 4% of the tablet weight. Suitable film-forming agents are well known to the art and include hydroxypropyl. methylcellulose, cationic methacrylate copolymers (dimethylaminoethyl methacrylate/methyl-butyl methacrylate copolymers—Eudragit® E—Röhm. Pharma), and the like. These film-forming agents may optionally contain colorants, plasticizers, and other supplemental ingredients.

The compressed tablets preferably have a hardness sufficient to withstand 8 Kp compression. The tablet size will depend primarily upon the amount of compound in the tablet. The tablets will include from 300 to 1100 mg of compound free base. Preferably, the tablets will include amounts of compound free base ranging from 400-600 mg, 650-850 mg, and 900-1100 mg.

In order to influence the dissolution rate, the time during which the compound containing powder is wet mixed is controlled. Preferably the total powder mix time, i.e. the time during which the powder is exposed to sodium hydroxide solution, will range from 1 to 10 minutes and preferably from 2 to 5 minutes. Following granulation, the particles are removed from the granulator and placed in a fluid bed dryer for drying at about 60° C.

It has been found that these methods produce sustained release formulations that provide lower peak plasma levels and yet effective plasma concentrations of compound for up to 12 hours and more after administration, when the compound is used as its free base, rather than as the more pharmaceutically common dihydrochloride salt or as another salt or ester. The use of free base affords at least one advantage: The proportion of compound in the tablet can be increased, since the molecular weight of the free base is only 85% that of the dihydrochloride. In this manner, delivery of an effective amount of compound is achieved while limiting the physical size of the dosage unit.

The oral sustained release Ranolazine dosage formulations of this invention are administered one, twice, or three times in a 24 hour period in order to maintain a plasma Ranolazine level above the threshold therapeutic level and below the maximally tolerated levels, which is preferably a plasma level of about 550 to 7500 ng base/mL in a patient. In a preferred embodiment, the plasma level of Ranolazine ranges about 1500-3500 ng base/mL.

In order to achieve the preferred plasma Ranolazine level, it is preferred that the oral Ranolazine dosage forms described herein are administered once or twice daily. If the dosage forms are administered twice daily, then it is preferred that the oral Ranolazine dosage forms are administered at about twelve hour intervals.

In another embodiment of the invention, Ranolazine may be incorporated into a pharmaceutical formulation for topical administration. This type of formulation typically contains a pharmaceutically acceptable carrier that is generally suited to topical drug administration and comprising any such material known in the art. Suitable carriers are well known to those of skill in the art and the selection of the carrier will depend upon the form of the intended pharmaceutical formulation, e.g., as an ointment, lotion, cream, foam, microemulsion, gel, oil, solution, spray, salve, or the like, and may be comprised of either naturally occurring or synthetic materials. It is understood that the selected carrier should not adversely affect Ranolazine or other components of the pharmaceutical formulation.

Suitable carriers for these types of formulations include, but are not limited to, vehicles including Shephard's™ Cream, Aquaphor™, and Cetaphil™ lotion. Other preferred carriers include ointment bases, e.g., polyethylene glycol-1000 (PEG-1000), conventional creams such as HEB cream, gels, as well as petroleum jelly and the like. Examples of suitable carriers for use herein include water, alcohols and other nontoxic organic solvents, glycerin, mineral oil, silicone, petroleum jelly, lanolin, fatty acids, vegetable oils, parabens, waxes, and the like. Particularly preferred formulations herein are colorless, odorless ointments, lotions, creams, microemulsions and gels.

Ointments are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's Pharmaceutical Sciences, 20^{th} Ed. (Easton, Pa.: Mack Publishing Company, 2000), ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols (PEGs) of varying molecular weight; again, reference may be had to Remington's, supra, for further information.

Lotions are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. A particularly preferred lotion formulation for use in conjunction with the present invention contains propylene glycol mixed with a hydrophilic petrolatum such as that which may be obtained under the trademark Aquaphor™ from Beiersdorf, Inc. (Norwalk, Conn.).

Creams containing the active agent are, as known in the art, viscous liquid or semisolid emulsions, either oil-in-water or water-in-oil. Cream bases are water-washable, and contain an oil phase, an emulsifier, and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic, or amphoteric surfactant.

Microemulsions are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, a surfactant (emulsifier), a co-surfactant (co-emulsifier), an oil phase, and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives, and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

Gel formulations are semisolid systems consisting of either small inorganic particle suspensions (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in the topical formulations of the invention. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients, and preservatives.

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in the formulation, e.g., anti-inflammatory agents, other analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In the preferred topical formulations of the invention, the Ranolazine is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Also, the pharmaceutical formulation may be sterilized or mixed with auxiliary agents, e.g., preservatives, stabilizers, wetting agents, buffers, or salts for influencing osmotic pressure and the like.

Sterile injectable solutions are prepared by incorporating Ranolazine in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Ranolazine Blockage of Na_{V}1.7 Ion Channels

### Materials and Methods

### Heterologous Expression: DNA Constructs and Transfection SCN9A Na⁺ Channel.

Human embryonic kidney (HEK293) cells stably transfected with cDNA encoding the α- and β1 subunits of SCN9A Na⁺ channel were purchased from Scottish Biomedical, Glasgow, United Kingdom. HEK293 cells were cultured in Dulbecco's modified Eagle's medium (DMEM) containing 10% fetal bovine serum and 1% penicillin and 1% streptomycin.

### Patch-Clamp Recording Technique

Membrane currents were recorded using the whole-cell patch clamp technique (18±1° C.). pCLAMP 10.0 software (Axon Instruments, Sunnyvale, Calif.) was used to generate voltage clamp protocols and acquire data, which were analyzed using pCLAMP 10.0 and Microcal Origin (MicroCal, Northampton, Mass.) software. During recording of Nav1.7 peak sodium current (I_{Na}), the extracellular bath solution contents were (in mM): NaCl 140, KCl 4, CaCl₂ 1.8, MgCl₂ 0.75, HEPES 5 (pH 7.4 after titration with NaOH). The intracellular pipette solution contents were (in mM): CsF 120, CsCl 20, EGTA 2, HEPES 5 (pH 7.4 after titration with CsOH). The Axopatch-200B patch clamp amplifier (Axon Instruments Inc., Sunnyvale, Calif.) was used to record I_{Na} and to measure cellular capacitance. Data were sampled at 20 kHz and filtered (8-pole Bessel) at 5 kHz. Series resistance (R₅) compensation was 70-80% and leak subtraction was not used.

### Sources and Administration of Drugs

Research grade Ranolazine (racemic mixture), R-Ranolazine and S-Ranolazine were synthesized by the Department of Bio-Organic Chemistry at CV Therapeutics, Inc (Palo Alto, Calif.) and dissolved in 0.1 N HCl to give stock solutions of 10 mM concentration. Further dilutions were freshly made in Tyrode solution on the day of an experiment.

### Statistical Analysis

Data are presented as mean±SEM. Concentration-response relations were fitted using the Hill equation, I_{drug}/I_{control}=1/[1+(D/IC₅₀)ⁿ], where I_{drug}/I_{control} is fractional block, D is drug concentration, IC₅₀ is the drug concentration that causes 50% block and n_{H} is the Hill coefficient. Statistical significance of differences was determined using Student's paired t-test and a p value of <0.05 was considered significant.

### Results

### Characterization of Sodium Channel Conductance in HEK293 Cells Stably Expressing Nav1.7 I_{Na}

Na⁺ currents are separated on the basis of their different sensitivities to TTX. Nav1.7 peak I_{Na} is reported to be sensitive to TTX with an IC₅₀ value of ˜3 nM (Zhou et al, JPET, 306: 498-504). To demonstrate the sensitivity Nav1.7 peak I_{Na} to TTX in our laboratory, cells were depolarized every 10 sec (0.1 Hz) from a holding potential of −100 mV to 0 mV for 50 msec. In each cell studied, after obtaining a baseline current recording in the absence of drug, perfusion of the experimental chamber was continued with Tyrode solution contained 300 nM TTX. Nav1.7 peak I_{Na} was completely blocked during exposure to TTX. Similar effects were observed in 3 different cells (data not shown).

### Block of Nav1.7 Peak I_{Na} by Ranolazine (Racemic Mixture) and its Enantiomers (R- and S-Ranolazine)

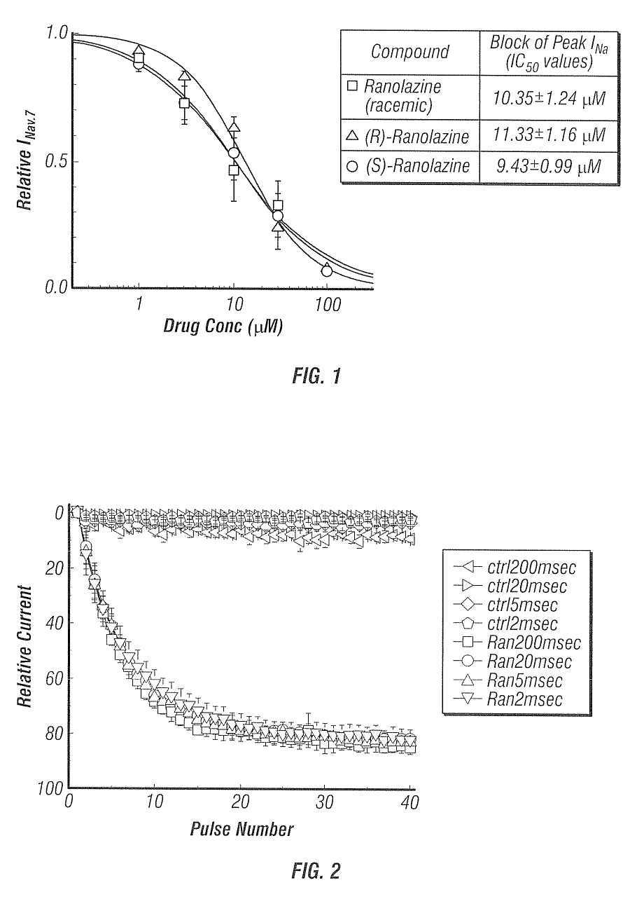

To determine the concentration-response relations of Ranolazine to inhibit Nav1.7 peak I_{Na}, individual cells expressing the SCN9A (Nav1.7) gene were depolarized every 10 sec (0.1 Hz) from a holding potential of −100 mV to 0 mV for a period of 50 msec. The magnitude of peak I_{Na} in the presence of increasing concentrations of Ranolazine (▪, 1 to 30 μM) or R-Ranolazine (▴, 1 to 100 μM) and S-Ranolazine (, 1 to 100 μM) was normalized to the respective control value in the absence of drug and plotted as relative current (FIG. 1). The IC₅₀ and n_{H} values for the block of Nav1.7 peak I_{Na} by Ranolazine, R-Ranolazine and S-Ranolazine are given in Table 3.

TABLE 3
Effect of Ranolazine on Nav1.7 Peak I_{Na}

| | | |
|---|---|---|
| Number of exp. | Mean* | ±SEM |

Ranolazine (μM)

| | | | |
|---|---|---|---|
| Control | 9 | 1 | 0 |
| 1 | 4 | 0.9114 | 0.02769 |
| 3 | 5 | 0.72745 | 0.08144 |
| 10 | 3 | 0.4692 | 0.12563 |
| 30 | 6 | 0.32702 | 0.097 |

R-Ranolazine (μM)

| | | | |
|---|---|---|---|
| Control | 6 | 1 | 0 |
| 1 | 3 | 0.92516 | 0.00966 |
| 3 | 4 | 0.82855 | 0.02324 |
| 10 | 3 | 0.63357 | 0.04231 |
| 30 | 3 | 0.23872 | 0.08601 |
| 100 | 4 | 0.07855 | 0.00813 |

S-Ranolazine (μM)

| | | | |
|---|---|---|---|
| Control | 4 | 1 | 0 |
| 1 | 4 | 0.87883 | 0.03003 |
| 3 | 4 | 0.72854 | 0.06445 |
| 10 | 5 | 0.53577 | 0.10721 |
| 30 | 4 | 0.28831 | 0.08653 |
| 100 | 2 | 0.06853 | 0.01647 |

Data: Data7_F (Ranolazine), IC50_H (R-Ranolazine, S-Ranolazine)
Model: Logistic
Equation: y = A2 + (A1 − A2)/(1 + (x/x0)\hat{ }p)
Weighting:
y No weighting

| | Ranolazine | | R-Ranolazine | | S-Ranolazine | |
|---|---|---|---|---|---|---|
| Chi\hat{ }2/DoF | 0.0015 | | | | | |
| R\hat{ }2 | 0.9862 | | | | | |
| A1 | 1 | 0 | 1 | 0 | 1 | 0 |
| A2 | 0 | 0 | 0 | 0 | 0 | 0 |
| x0 | 10.3585 | 1.24518 | 11.33403 | 1.16748 | 9.43309 | 0.99646 |
| p | 0.8368 | 0.09364 | 1.21171 | 0.11868 | 0.94091 | 0.08891 |

*Amplitude of I_{Na} - Frication of control

### Open State Block of Nav1.7 by Ranolazine

To understand whether Ranolazine preferentially binds to the open or inactivated states of Nav1.7 I_{Na}, individual HEK293 cells were depolarized with trains of 40 pulses from a holding potential of −100 mV to 0 mV for a period of 2, 5, 20, or 200 msec (step duration) with the same pulse applied every 200 msec (i.e., at a rate of 5 Hz). The magnitude of peak I_{Na} in the absence (control) or presence of 100 μM Ranolazine was normalized to peak I_{Na} value recorded in response to first depolarizing step (pulse 1). Without drug, repetitive pulses produce little (˜1 to 8%) or no reduction of the peak currents (FIG. 2, open symbols). If Ranolazine preferentially binds to the open state, significant block of peak I_{Na} will be observed irrespective of the duration of the depolarizing step (2, 5, 20 or 200 msec). A depolarizing voltage step of 2 or 5 msec in duration is too brief to allow the channel to transition from open to inactivated states, whereas a depolarizing voltage step of 200-msec duration will allow channels to transition from open to inactivated states.

As shown in FIG. 2 (closed symbols), depolarizing HEK293 cells expressing Nav1.7 to 0 mV for a period of 2, 5, 20 or 200 msec in the presence of 100 μM Ranolazine caused a significant block (˜82.72±0.71%) of peak I_{Na} at the end of the pulse train (FIG. 2, filled symbols; pulse 40). The percent block of peak I_{Na} by 100 μM Ranolazine was independent of the duration of the depolarizing step (2, 5, 20 or 200 msec). The finding that reduction of peak I_{Na} by 100 μM Ranolazine was independent of the duration of the depolarizing step indicates that the drug interacts with the open state of the Na⁺ channel Nav1.7.

### Example 2

### Ranolazine Blockage of Na_{V}1.7 and Na_{V}1.8 Sodium Currents

In this study we show that ranolazine inhibits Nav1.7 and Nav1.8 Na⁺ channels. These channels are present in peripheral pain-sensing neurons and are reported to play an important role in the etiology of neuropathic pain. Ranolazine inhibited hNav1.7 and rNav1.8 Na⁺ channels in a voltage- and use (frequency)-dependent manner. Ranolazine did not alter the activation voltage range of either Nav1.7 or Nav1.8 I_{Na}, or the voltage at which half-maximal activation (V_{1/2}) of current occurred. However, ranolazine caused a concentration-dependent hyperpolarizing shift of the inactivation voltages of both currents.

### Methods

### Expression of Sodium Channels.

HEK293 cells stably expressing the hNav1.7 (α-subunit) along with a human β₁ subunit were purchased from Scottish-Biomedical, Glasgow, UK. Cells were continuously maintained using MEM (Gibco-Invitrogen, Carlsbad, Calif.) supplemented with 10% heat inactivated fetal bovine serum, 1% penicillin-streptomycin, 600 μg/mL geneticin (Gibco-Invitrogen), 2 μg/mL blastocydin (Calbiochem, NJ, USA), and were incubated at 37° C. in an atmosphere of 5% CO₂ in air.

Transient or stable expression of Nav1.8 I_{Na} in heterologous expression systems has been shown to be problematic (John et al, 2004a). Therefore, in this study, recombinant ND7-23 (rat DRG/mouse neuroblastoma hybrid) cells stably expressing the rNav1.8 were purchased from Millipore (UK) limited, Cambridge, UK. It has been reported that ND7-23 cells also express a TTX-S I_{Na} that has rapid kinetics, but the molecular identity of these Na⁺ channels is still not clear (Dunn et al., 1991; John et al., 2004b). Cells were maintained using DMEM (Gibco-Invitrogen) supplemented with 10% fetal bovine serum, 1% L-glutamine, 1% non-essential amino acids, 1% penicillin-streptomycin, 400 μg/mL geneticin (Gibco-Invitrogen), and were incubated at 37° C. in an atmosphere of 5% CO₂ in air.

### Solutions and Chemicals

For recording hNav1.7 I_{Na}, HEK293 cells were superfused with an extracellular solution containing (in mM): 140 NaCl, 3KCl, 10 HEPES, 10 glucose, 1 MgCl₂, 1 CaCl₂, pH 7.4 (with NaOH). Patch pipettes were filled with an internal solution containing (in mM): 140 CsF, 10 NaCl, 1 EGTA, 10 HEPES, pH 7.3 (with CsOH). For recording endogenous I_{Na} in ND7-23 cells or rNav1.8 I_{Na}, cells were superfused with an extracellular solution containing (in mM): 140 NaCl, 5 HEPES-Na, 1.3 MgCl₂, 1 CaCl₂, 11 glucose, 4.7 KCl, pH 7.4. Patch pipettes were filled with an internal solution containing (in mM): 120 CsF, 10 HEPES, 10 EGTA, 15 NaCl, pH 7.25. To determine the use-dependence of drug block of rNav1.8, experiments were performed using a test potential of +50 mV (at which the Na⁺ current is outward) and an extracellular solution containing (in mM): 65 NaCl, 85 choline Cl, 2 CaCl₂, 10 HEPES, pH 7.4 (with tetramethylammonium hydroxide). Patch pipettes were filled an internal solution containing (in mM): 100 NaF, 30 NaCl, 10 EGTA, 10 HEPES, pH 7.2 (with CsOH). The reversed Na⁺ gradient was employed to minimize series resistant artifacts, which are less serious with outward than with inward I_{Na} flow.

Unless otherwise mentioned, patch-clamp studies using ND7-23 cells were performed in the continuous presence of 300 nM TTX to block the endogenous TTX-S I_{Na} (Ogata and Tatebayashi, 1993; Roy and Narahashi, 1992). Research grade ranolazine was synthesized by the Department of Bio-Organic Chemistry at CV Therapeutics, Inc (Palo Alto, Calif.) and TTX was purchased from Sigma (St. Louis, Mo.). Ranolazine was dissolved in 0.1 N HCl to give a stock solution of 10 mM and further dilutions were freshly made in Tyrode solution on the day of the experiments. TTX was dissolved in distilled water.

### Electrophysiological Technique and Data Acquisition

Whole-cell I_{Na} was recorded as described by (Hamill et al., 1981) using an Axopatch 200B amplifier (Molecular Devices, Sunnyvale, USA). Signals were filtered at 5 kHz and sampled at 20 kHz. Patch pipettes were formed from borosilicate glass (World Precision Instruments, Sarasota, USA) using a micropipette puller (Dagan Corporation, Minneapolis, USA). The offset potential was zeroed before the pipette was attached to the cell and the voltages were not corrected for the liquid junction potential. In all recordings, 75-80% of the series resistance compensation was achieved, thus yielding a maximum voltage error of ˜5 mV and leak currents were cancelled by P/−4 subtraction. pCLAMP 10.0 software (Molecular Devices) was used to generate voltage clamp protocols and acquire data. Cells were held at a membrane potential of −100 or −120 mV and were dialyzed with pipette solution for 5-7 minutes before current was recorded, to avoid time-dependent shifts in Na⁺ channel gating within the first several minutes after patch rupture. In all experiments, the temperature of experimental solutions was maintained at 20±1° C. using a CL-100 bipolar temperature controller (Warner Instruments, Hamden, USA).

Data were analyzed using Clampfit and Microcal Origin (MicroCal, Northampton, USA) software. Results are expressed as mean±S.E.M., and n refers to number of cells. All experiments were repeated on at least 2 different experimental days. Statistical significance of differences between responses of a cell in the absence and presence of drug was determined using the Student t-test, with P<0.05 indicating statistical significance.

Concentration-response relations were fit using the Hill equation,

I_{drug}/I_{control}=1/[1+(D/IC₅₀)ⁿ_{H}],

where I_{drug}/I_{control} is fractional block, D is drug concentration, IC₅₀ is the drug concentration that causes 50% block and n_{H} is the Hill coefficient.

The voltage dependence of activation was determined using 50-msec depolarizing pulses from a holding potential of −120 or −100 mV to test potentials ranging from −80 to +40 mV, in 5 mV increments. To determine the voltage dependence of channel activation, Na⁺ conductance (G_{Na}) was calculated from the peak current (I_{Na}), using the equation:

G_{Na}=I_{Na}/(V−Vrev),

where V is the test pulse potential and Vrev is the calculated reversal potential. Normalized Na⁺ conductance was plotted against test pulse potential and fit to a Boltzmann equation:

G/Gₘₐₓ=1/[1+exp(V_{1/2}−V/k)],

where G is the measured conductance, Gₘₐₓ is the maximal conductance, V_{1/2} is the membrane potential at which the half-maximal channel open probability occurs and k is the slope of the curve. For assessing the voltage dependence of steady-state inactivation, prepulses ranging from −120 to 0 mV (for hNav1.7 I_{Na}) or −100 to +20 mV (for rNav1.8 I_{Na}) were applied for a period of 1 sec, followed by a 50-msec depolarizing step to 0 mV (for hNav1.7 I_{Na}) or to +20 mV (rNav1.8 I_{Na}).

The peak current (I) was normalized relative to the maximal value (Iₘₐₓ) obtained at a holding potential (Vₕ) of −100 or −120 mV and plotted against the conditioning pulse potential. Data were fit to a Boltzmann equation:

I/Iₘₐₓ=1/[1+(exp(V−V_{1/2}/k)],

where V is the membrane potential during the pre-pulse, V_{1/2} the potential at which the half-maximal channel inactivation occurs and k is the slope factor. For assessing the voltage dependence of steady-state slow inactivation, prepulses ranging from −120 to −10 mV (for hNav1.7 I_{Na}) or −100 to −10 mV (for rNav1.8 I_{Na}) were applied for a period of 10 msec, followed by a 100-msec hyperpolarizing step to −160 (for hNav1.7) or −140 mV (for rNav1.8) and then stepped to 0 (for hNav1.7 I_{Na}) or +20 mV (rNav1.8 I_{Na}) for a period of 50-msec to measure the available current. The brief 100-msec hyperpolarizing step was employed to allow channels (both without and with drug bound) to recovery from fast, but not slow-inactivation. Data from the voltage dependence of steady-state slow inactivation were fit with a modified Boltzmann equation (Carr et al. (2003). Neuron; 39:793-806 and Vilin et al. (2001) Cell Biochem Biophys, 35:171-190):

I/Iₘₐₓ=(1−I_{resid})/[1+exp(−(V−V_{1/2})/k)],

where I_{resid} is the residual (noninactivating) fraction of the current.

To estimate the extent of block of inactivated channels by ranolazine, an indirect approach based on the concentration-dependence of the shift of the steady-state inactivation curve was used ((Bean et al., 1983), see equation below).

ΔV_{1/2}=k In[(1+(D/IC_{50R}))/(1+([D/K_{I}))]

where ΔV_{1/2} is the shift in midpoint of the steady-state inactivation curve, k is the slope factor of the steady-state inactivation curve derived from a Boltzmann fit, [D] is the concentration of ranolazine applied, IC_{50R} is the IC₅₀ value for resting channels, K_{I} is the dissociation constant for block of inactivated channels by ranolazine.

Recovery from inactivation was measured with a standard two-pulse protocol of 50 msec in duration with an incremental time delay of 1 msec to 8 sec between the two pulses (holding potential=−100 mV; test potential=−20 mV (hNav1.7 I_{Na}) or +20 mV (rNav1.8 I_{Na}). The peak current elicited by the second pulse (I) was normalized relative to the current elicited by the first pulse (I₀). The duration of every cycle of the double pulse protocol was 20 sec. I/I₀ was plotted against the time delay between the two pulses and fit to a double or triple exponential function,

I/I₀=[A_{F}*exp(−t/τ_{F})]+A_{S}*exp(−t/τ_{S})+A_{∞},

where t=recovery time interval, τ_{F} and τ_{S}=fast and slow time constants, A_{F} and A_{S}=relative amplitude of the fast and slow recovery component, and A_{∞} is the relative amplitude of the steady-state component), or

I/I₀=[A_{F}*exp(−t/τ_{F})]+A_{I}*exp(−t/τ_{I})+A_{S}*exp(−t/τ_{S})+A_{∞},

where t=recovery time interval, τ_{F}, τ_{I} and τ_{S}=fast, intermediate and slow time constants, A_{F}, A_{I} and A_{S}=relative amplitude of the fast and slow recovery component, and A_{∞} is the relative amplitude of the steady-state component.

### Results

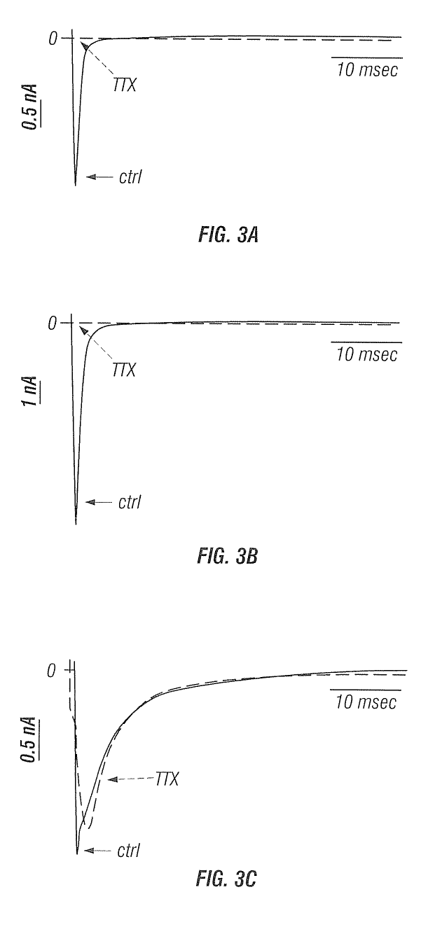

FIG. 3 shows the effect of 300 nM TTX on HEK293 cells stably expressing hNav1.7+β1 subunits (FIG. 3A) and untransfected ND7-23 cells (FIG. 3B) or ND7-23 cells stably expressing rNav1.8 Na⁺ channels (ND7-23/rNav1.8; FIG. 3C). TTX (300 nM) completely blocked the hNav1.7 I_{Na} in HEK293 and endogenous I_{Na} in ND7-23 cells. In contrast, 300 μM TTX caused a minimal block of rNav1.8 I_{Na}, confirming previous reports of the resistance of rNav1.8 to the toxin. (Ogata and Tatebayashi, 1993; Roy and Narahashi, 1992).

### Ranolazine Blocks Recombinant Human and Native Rat Na1.7 and Nav1.8 Currents.

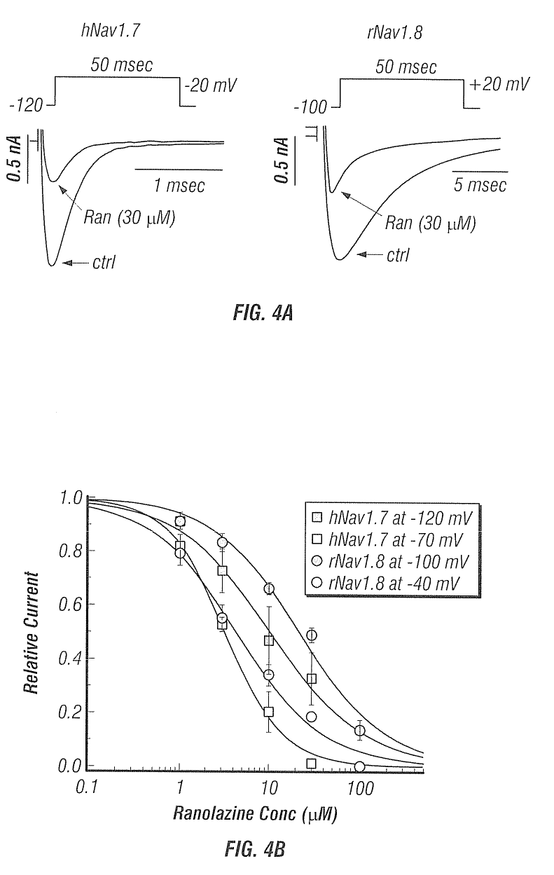

The application of 30 μM ranolazine to either HEK293 cells stably expressing hNav1.7 or ND7-23 cells stably expressing rNav1.8 Na⁺ channels produced a significant reduction of peak current (FIG. 4A) and suggested a considerable acceleration of the rate of inactivation (transition from open to inactivated states). To quantify the changes in I_{Na} decay rates (in the absence and presence of 30 μM ranolazine) the current traces (hNav1.7 and rNav1.8) were fit with single exponentials. At −20 mV the decay of hNav1.7 currents for control conditions and in the presence of 30 μM ranolazine had time constants of 1.51±0.31 and 0.68±0.15 msec (n=4 cells, p<0.05), respectively. Similarly, at +20 mV the decay of rNav1.8 currents for control conditions and in the presence of 30 μM ranolazine had time constants of 3.40±0.13 and 1.60±0.04 msec (n=4 cells, p<0.05), respectively.

Ranolazine caused a concentration-dependent block of hNav1.7 and rNav1.8 at holding potentials of −120 or −100 mV, respectively (FIG. 4B, Table 4, V₀). When the holding potential in experiments was set at a voltage close to the midpoint of the voltage-dependent steady-state inactivation relationship (voltage at which 50% of channels are inactivated, V_{0.5}) for each channel (−70 mV for Nav1.7 and −40 mV for Nav1.8), the concentration-response relationship for ranolazine block of I_{Na} was shifted to the left (i.e., to lower ranolazine concentrations) (FIG. 4B, Table V_{0.5}). Ranolazine also blocked the endogenous TTX-S I_{Na} in ND7-23 cells in a concentration-dependent manner (See Table 4 for IC₅₀ value).

TABLE 4
Block of hNav1.7, rNav1.8 and TTX-S by Ranolazine.

IC₅₀ value (μM), [Hill Coefficient]

| Nav Isoform | V₀ | V_{0.5} |
|---|---|---|
| Nav1.7 | 10.36 ± 1.25 | 3.25 ± 0.17 |
| | [0.84 ± 0.09] | [1.33 ± 0.08] |
| Nav1.8 | 21.53 ± 3.01 | 4.33 ± 0.52 |
| | [0.90 ± 0.11] | [0.89 ± 0.09] |
| TTX-S | 9.05 ± 0.56 | N.T. |
| | [1.15 ± 0.08] | |

V_{0,} Holding potential of −120 mV (Nav1.7) or −100 mV (Nav1.8)
V_{0.5.} Holding potential of −70 mV (Nav1.7) or −40 mV (Nav1.8)

For these experiments, endogenous I_{Na} was recorded in the absence of 300 nM TTX. Half-maximal inhibitory concentrations (IC₅₀ values) derived from fits of data plotted as relative reduction of peak I_{Na} versus drug (ranolazine) concentration (FIG. 4) are summarized in Table 4. The Hill coefficients of the relationships between ranolazine concentration and reduction of peak I_{Na} were near one (FIG. 4B), indicating an 1:1 stoichiometry of drug and Na⁺ channel interaction.

### Voltage Dependence of Activation in the Presence of Ranolazine

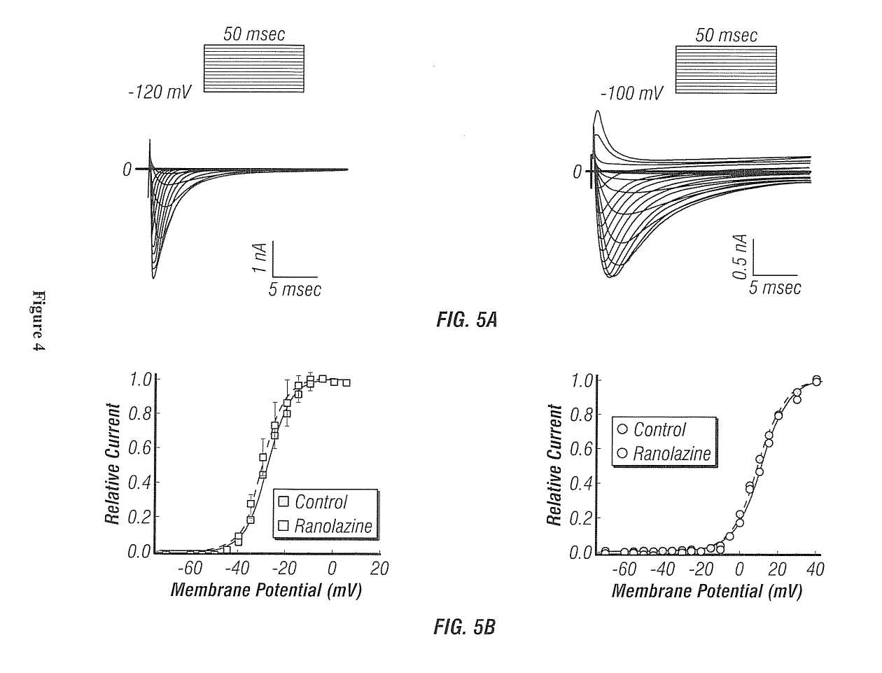

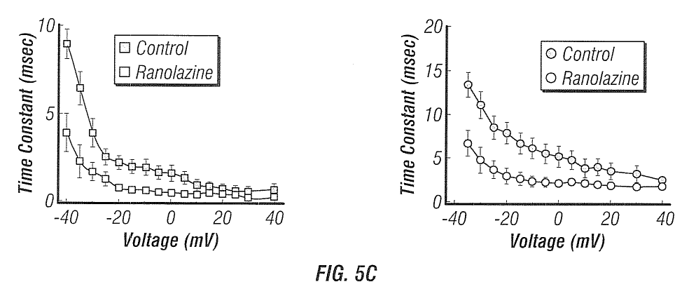

Current-voltage (I-V) relationships for hNav1.7 and rNav1.8 I_{Na} were determined in the absence and presence of 10 μM ranolazine using a series of 50-msec depolarizing steps from a holding potential of −120 (for hNav1.7) or −100 (rNav1.8) mV with an interpulse interval of 10 sec. FIG. 5A shows the voltage clamp protocols and representative current traces recorded from a HEK293 cell stably expressing hNav1.7 (left panel) and from ND7-23/rNav1.8 I_{Na} (right panel, recorded in the presence of 300 nM TTX), respectively. From the peak amplitude of I_{Na} measured, sodium conductance (G_{Na}) was calculated (see Methods for details) and the voltage-dependence of G_{Na} was plotted in the absence (▪, hNav1.7; , rNav1.8, FIG. 5B) and presence (□, hNav1.7; ∘, rNav1.8, FIG. 5B) of 10 μM ranolazine.

The values of mean half-maximal voltage (V_{1/2}) for activation and the slope (k) factors of the relationships in the absence (control) and presence of ranolazine are shown in Table 5. Ranolazine did not significantly shift the voltage range across which channel activation occurred (FIG. 5, Table 5, Activation). FIG. 5C shows the decay of the hNav1.7 (left panel) and rNav1.8 (right panel) I_{Na} (current traces described in FIG. 5C) in the absence () and presence of 10 μM (∘) ranolazine fit to a single exponential equation. Ranolazine caused a significant effect to decrease the time constants of current decay at voltages between −40 to +5 mV for hNav1.7 and −35 to +30 mV for rNav1.8, respectively. (Table 5, Inactivatoin)

TABLE 5
Comparative Activation and Inactivation Parameters of hNav1.7 and
rNav1.8 in the Absence (control) and Presence of 10 mM Ranolazine.

| | | hNav1.7 | | rNav1.8 | |
|---|---|---|---|---|---|
| | | | k | | K |
| | | V_{1/2} (mV) | (mV/e-fold) | V_{1/2} (mV) | (mV/e-fold) |
| Activation | Control | −32.65 ± 2.16 | 4.84 ± 0.38 | 8.72 ± 3.51 | 11.23 ± 1.62 |
| | (▪, ) | | | | |
| | Ranolazine | −33.96 ± 2.06 | 4.77 ± 0.32 | 4.71 ± 2.58 | 8.55 ± 1.62 |
| | 10 μM (□, ∘) | | | | |
| Inactivation | Control | −74.06 ± 2.96 | 4.67 ± 0.16 | −37.43 ± 3.13 | 7.58 ± 1.03 |
| | (▪) | | | | |
| | Ranolazine | | | | |
| | 1 μM | −78.97 ± 3.09 | 4.80 ± 0.21 | −42.75 ± 3.93 | 7.38 ± 0.93 |
| | 3 μM | −84.44 ± 3.64* | 4.76 ± 0.11 | −47.61 ± 4.56* | 7.14 ± 0.61 |
| | 10 μM | −86.99 ± 2.86* | 4.56 ± 0.1 | −57.88 ± 5.73* | 7.59 ± 0.79 |
| | (□) | | | | |
| | 30 μM | −89.07 ± 5.41* | 5.15 ± 0.31 | −59.52 ± 2.18* | 7.59 ± 1.09 |

### Voltage Dependence of Steady-State Fast, Intermediate and Slow Inactivation in the Presence of Ranolazine

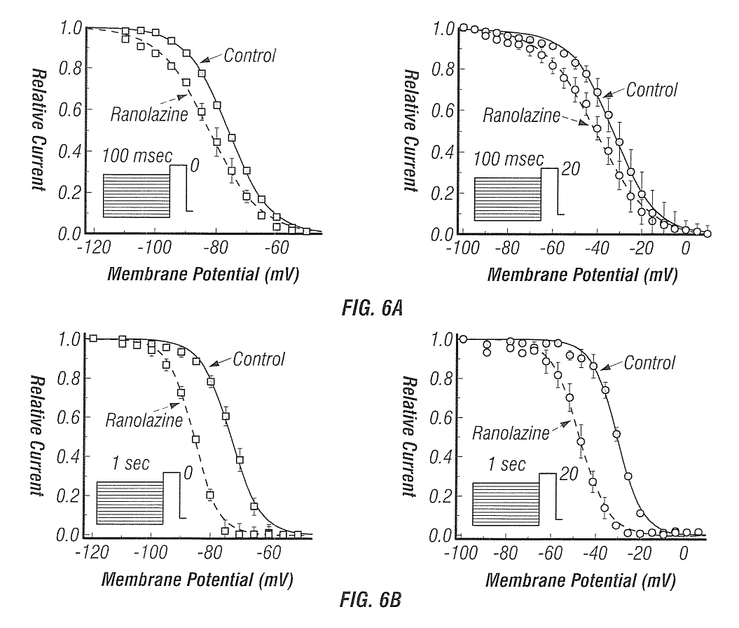

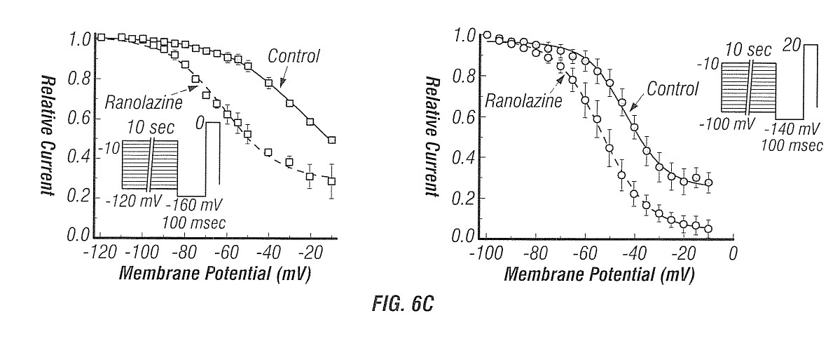

Results of experiments to determine the voltage dependence of steady-state fast, intermediate and slow inactivation of hNav1.7 (left panels) and rNav1.8 (right panels) I_{Na} are shown in FIG. 6. FIG. 6A shows voltage-clamp protocols and summary results of experiments for steady-state fast inactivation of hNav1.7 and rNav1.8 (inactivating prepulse of 100 msec) in the absence (▪, hNav1.7; , rNav1.8) and presence of 10 μM ranolazine (□, hNav1.7; ∘, rNav1.8). Ranolazine caused a significant (p<0.05) leftward shift in the V_{1/2} of fast-inactivation without affecting the slope (k) factor of hNav1.7, and a minimal (p=0.15) leftward shift in the V_{1/2} of fast-inactivation without affecting the slope (k) factor of rNav1.8 I_{Na} (see figure legends for values). FIG. 6B shows voltage-clamp protocols and summary results of experiments for steady-state intermediate inactivation of hNav1.7 and rNav1.8 (inactivating prepulse of 1 sec) in the absence (▪, ) and presence of 10 μM ranolazine (□, ∘).

Ranolazine caused a concentration-dependent (1-30 μM) leftward shift in the V_{1/2} of intermediate inactivation without affecting the slope (k) factor for hNav1.7 (n=4 cells at each concentration) and rNav1.8 (n=4-5 cells at each concentration) I_{Na} (Table 5, Inactivation). The data for midpoints of activation and steady-state inactivation for control conditions (hNav1.7 and rNav1.8) in the present study are comparable to values found previously for ND7-23/rNav1.8 and native TTX-S and TTX-R currents in DRG neurons. Cummins et al (1997) J Neurosci, 17:3503-14 and John et al. (2004) Neuropharmacology 46:425-38.

To test the voltage dependence of the steady-state slow inactivation process, the pulse protocol shown in FIG. 6C was employed for both hNav1.7 and rNav1.8. Using this protocol, slow inactivation (physiological) became evident at potential of −80 mV and −75 mV for hNav1.7 and rNav1.8, respectively. However, slow-inactivation was only 50 and 70% complete at the maximum conditioning test pulse of −10 mV. FIG. 6C shows voltage-clamp protocols and summary results of experiments for steady-state slow inactivation of hNav1.7 and rNav1.8 (inactivating prepulse of 10 sec) in the absence (▪, ) and presence of 10 μM ranolazine (□, ∘). Ranolazine caused a significant (p<0.05) leftward shift in the V_{1/2} of slow inactivation without affecting the slope (k) factor of hNav1.7 and rNav1.8 I_{Na} (see figure legends for values).

The ranolazine-induced shift in the mid-point (V_{1/2}) of inactivation (FIG. 6) and voltage-dependent block of hNav1.7 and rNav1.8 (FIG. 4, Table 4, at V_{1/2} holding potential IC₅₀ values) suggest that ranolazine might be interacting with the inactivated states of these channels. To estimate the extent of block of inactivated channels by ranolazine, an indirect approach based on the concentration-dependence of the shift of the steady-state inactivation curve 28 was used (K_{dr} and K_{di} values, calculated as described in Methods). Estimates of dissociation constants for ranolazine to bind to rested (K_{dr}) and inactivated (K_{di}) states of hNav1.7 and rNav1.8 channels were found to be 12.12 and 22.84 μM and 0.47 and 0.64 μM, respectively.

### Development of Inactivation in the Presence of Ranolazine

Ranolazine caused a hyperpolarizing shift in the voltage dependence of Nav1.7 and 1.8 I_{Na} availability (FIG. 6, Table 5 and the estimated K_{di} values using Bean equation), suggesting that the drug interacts with the inactivated state of these Na⁺ channels. To better understand the interaction of ranolazine with Nav1.7 and Nav1.8 channels, the rate of development of slow inactivation was determined by depolarizing the cells to −40 and −20 (hNav1.7) or −20 and +20 mV for a variable interval (0.1 to 10-sec) to allow development of block. A 20-msec hyperpolarizing step was inserted to allow recovery of unbound channels from fast inactivation before a standard test pulse to assess channel availability.

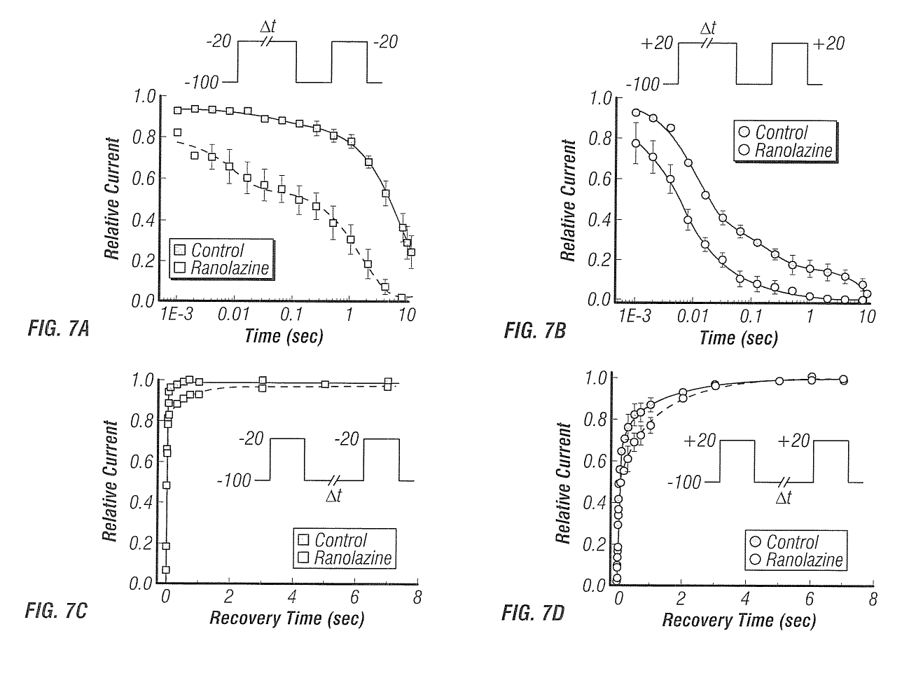

The time dependence of development of inactivation of hNav1.7 (−20 mV, FIG. 7A, n=4-5 cells) and rNav1.8 (+20 mV, FIG. 7B, n=4-5 cells) I_{Na} in the absence (▪) and presence (□) of 30 μM ranolazine is shown in FIG. 7. For control conditions, the progressive decay of currents with increasing conditioning pulse duration reflects entry of channels into inactivated states. The development of slow inactivation of hNav1.7+β1 and rNav1.8 channels could be fit with double and triple exponential functions, respectively (see Table 6, control, Development of slow inactivation).

As shown previously, (Vijayaragavan et al (2001) J. Neurosci 21:7909-18) the onset of slow inactivation of Nav1.8 channels is rapid when compared to Nav1.7 channels (˜fourfold, see Table 6, control, τ_{F}=10.78 and 43.97 msec for Nav1.8 and Nav1.7 channels, respectively). The rate of development of slow inactivation was 2-5 fold faster in the presence of ranolazine (30 μM) (see Table 6, ranolazine, Development of inactivation). The time constants for development of inactivation of hNav1.7 (n=4 cells) and rNav1.8 (n=5 cells) I_{Na} with a depolarizing prepulse to −40 mV (hNav1.7) or −20 mV (rNav1.8) in the absence and presence of 30 μM ranolazine are given in Table 6. The rate of development of slow inactivation was 4-10 fold faster in the presence of ranolazine (30 μM) (see Table 6, ranolazine, Development of inactivation at −40 (hNav1.7) and −20 mV (rNav1.8), respectively).

Recovery from Ranolazine Block

The effects of ranolazine on recovery from inactivation of hNav1.7 and rNav1.8 were assessed with a standard two-pulse protocol as described in Methods. The time dependence of recovery from inactivation of hNav1.7 (n=5 cells) and rNav1.8 (n=5 cells) I_{Na} in the absence (□) and presence (▪) of 30 μM ranolazine is shown in FIG. 7. For control conditions, recovery from inactivation of hNav1.7 I_{Na} (FIG. 7C, repolarizing potential=−100 mV) could be fit with a double exponential equation, with fast (τ_{F}) and slow time constants, (τ_{S}), respectively. In contrast, recovery from inactivation of rNav1.8 I_{Na} was slow (FIG. 7D), and could be better fit with three exponentials. The time course of recovery from inactivation of rNav1.8 I_{Na} (FIG. 7D, repolarizing potential=−100 mV) had fast (τ_{F}), intermediate (τ_{I}) and slow (τ_{S}) time constants.

As summarized in Table 6 (Recovery from inactivation at −100 mV), the fast component (τ_{F}) of hNav1.7 I_{Na} recovery from inactivation was not different in the absence and presence of 30 μM ranolazine, whereas the slow component (τ_{S}) was significantly (p<0.05) slowed in the presence of 30 μM ranolazine (see Table 6, hNav1.7, Recovery from inactivation). The fast (τ_{F}), intermediate (τ_{I}) and slow (τ_{S}) components of rNav1.8 I_{Na} recovery from inactivation were significantly (p<0.05) slowed in the presence of 30 μM ranolazine (see Table 6, rNav1.8, Recovery from inactivation).

The time dependence of recovery from inactivation of hNav1.7 (n=5 cells) and rNav1.8 (n=4 cells) I_{Na} with a depolarizing prepulse to −40 mV (hNav1.7) or −20 mV (rNav1.8) in the absence and presence of 30 μM ranolazine are plotted in Table 6. As summarized in Table 6 (Recovery from inactivation at −80 mV), the fast (τ_{F}) and slow (τ_{S}) components of hNav1.7 I_{Na} recovery from inactivation were significantly (p<0.05) slowed in the presence of 30 μM ranolazine. Similarly, ranolazine (30 μM) caused a significant (p<0.05) slowing of the fast (τ_{F}), intermediate (τ_{I}) and slow (τ_{S}) components of rNav1.8 I_{Na} recovery from inactivation (see Table 6, rNav1.8, Recovery from inactivation at −80 mV).

TABLE 6
Development of slow inactivation and recovery from inactivation
parameters of hNav1.7 and rNav1.8 in the absence (control) and presence of 30 μM
ranolazine.

| | | Development of slow inactivation | | Recovery from inactivation | |
|---|---|---|---|---|---|
| | | Control | Ranolazine | Control | Ranolazine |
| | | (at −20 mV) | | (at −100 mV) | |
| hNav1.7 | A_{F} | 0.16 ± 0.08 | 0.24 ± 0.02* | 0.82 ± 0.04 | 0.73 ± 0.03* |
| | A_{S} | 0.82 ± 0.03 | 0.56 ± 0.01* | 0.12 ± 0.02 | 0.16 ± 0.02 |
| | τ_{F} | 43.97 ± 15.76 | 9.14 ± 2.49* | 1.94 ± 0.31 | 2.15 ± 0.22 |
| | τ_{S} | 7372.66 ± 654.66 | 1657.71 ± 180.23* | 54.85 ± 3.53 | 546.44 ± 171.03* |
| | | (at −40 mV) | | (at −80 mV) | |
| | A_{F} | 0.11 ± 0.02 | 0.30 ± 0.02* | 0.88 ± 0.01 | 0.75 ± 0.02* |
| | A_{S} | 0.89 ± 0.02 | 0.67 ± 0.02* | 0.12 ± 0.01 | 0.19 ± 0.02* |
| | τ_{F} | 39.12 ± 4.91 | 23.99 ± 2.92* | 30.31 ± 1.01 | 37.72 ± 3.51* |
| | τ_{S} | 7923.74 ± 850.36 | 1845.75 ± 129.03* | 2279.19 ± 609.28 | 4724.79 ± 1301.69* |
| | | (at +20 mV) | | (at −100 mV) | |
| rNav1.8 | A_{F} | 0.59 ± 0.02 | 0.53 ± 0.07 | 0.37 ± 0.01 | 0.56 ± 0.02* |
| | A_{I} | 0.23 ± 0.02 | 0.25 ± 0.07 | 0.27 ± 0.02 | 0.21 ± 0.06 |
| | A_{S} | 0.18 ± 0.04 | 0.09 ± 0.02* | 0.32 ± 0.01 | 0.19 ± 0.05* |
| | τ_{F} | 10.78 ± 1.05 | 5.59 ± 1.08* | 10.19 ± 0.76 | 19.62 ± 1.16* |
| | τ_{I} | 217.14 ± 57.43 | 34.11 ± 14.14* | 123.70 ± 17.92 | 371.16 ± 127.40* |
| | τ_{S} | 7116.63 ± 822.32 | 703.24 ± 373.82* | 1327.78 ± 137.63 | 2184.78 ± 488.76* |
| | | (at −20 mV) | | (at −80 mV) | |
| | A_{F} | 0.62 ± 0.02 | 0.52 ± 0.05* | 0.51 ± 0.04 | 0.39 ± 0.07* |
| | A_{I} | 0.1 ± 0.02 | 0.30 ± 0.06* | 0.29 ± 0.04 | 0.36 ± 0.07 |
| | A_{S} | 0.20 ± 0.05 | 0.18 ± 0.03 | 0.14 ± 0.01 | 0.25 ± 0.03* |
| | τ_{F} | 29.55 ± 4.72 | 22.75 ± 2.31* | 15.55 ± 3.30 | 25.04 ± 5.19* |
| | τ_{I} | 334.23 ± 89.43 | 98.08 ± 13.65* | 389.01 ± 83.41 | 701.39 ± 44.00* |
| | τ_{S} | 5304.62 ± 1302.11 | 1203.00 ± 337.50* | 2522.03 ± 222.96 | 3666.15 ± 491.92* |

Data were recorded using voltage-clamp protocols described in FIG. 5 and fitted with double or triple exponential equations.
*p < 0.05.

### Use-Dependent Block by Ranolazine

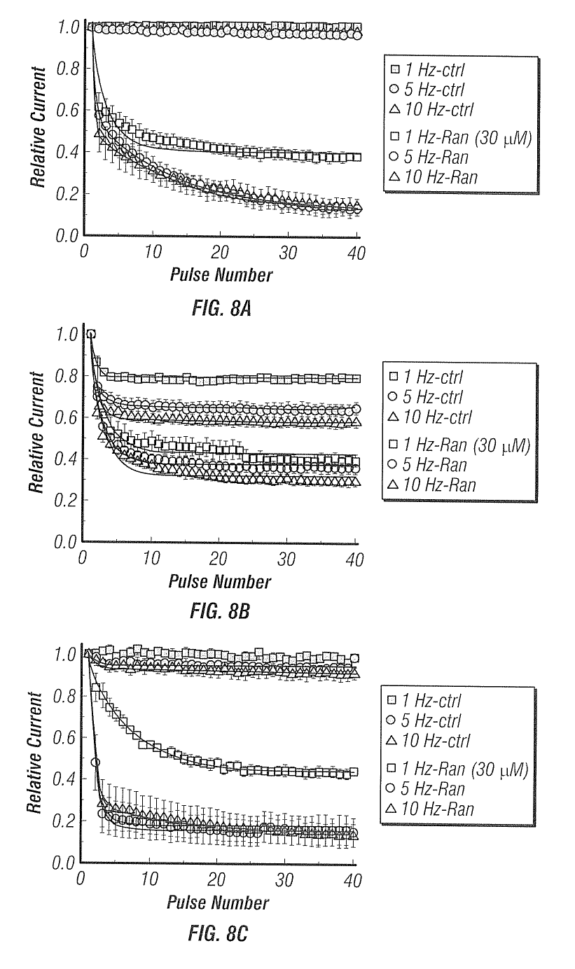

To study the use-dependent block of hNav1.7, rNav1.8 and TTX-S I_{Na} by ranolazine, a series of 40 short repetitive impulses (10 msec in duration) to −20 mV (for hNav1.7 and TTX-S I_{Na}) or to +50 mV (for rNav1.8 I_{Na}) from a holding potential of −100 mV at were applied rates of 1, 5 and 10 Hz. The amplitude of current evoked by the 40^{th} impulse was normalized to that of the current evoked by the first impulse. The short depolarizing pulse duration of 10 msec was chosen to approximate the somatic action potential duration of C fibers (0.6-7.4 msec; (Harper and Lawson, 1985). For hNav1.7 and TTX-S I_{Na}, pulsing frequencies up to 10 Hz had small effects on the amplitude of currents (FIGS. 8A and 8C, filled symbols), suggesting that the channels recovered rapidly from inactivation (τ_{S}=˜50 msec, Table 6) and could cycle quickly through open, closed and inactivated confirmations at these tested frequencies (Hille, 1977; Ragsdale et al., 1994; Roy and Narahashi, 1992; Vijayaragavan et al., 2001).

In contrast, rNav1.8 in control conditions showed a reduction in amplitude that depended on stimulating frequency (FIG. 8B, filled symbols). This frequency-dependent reduction in I_{Na} amplitude suggests that rNav1.8 Na⁺ channels in ND7-23 cells recover slowly from inactivation (τ_{S}=˜847 msec, Table 6). Ranolazine (30 μM) caused a frequency-dependent reduction (p<0.05, n=4-5 cells, each) in amplitude of hNav1.7, rNav1.8 and TTX-S I_{Na}, indicating marked use-dependent block. At the lowest stimulating frequency (1 Hz, □), ˜20-40% (depending on the channel isoform) of available channels were readily blocked by the drug. Increasing the stimulation frequency from 1 to 5 (∘) or 10 Hz (Δ) revealed additional rapidly equilibrating channel block, although block appeared to saturate at 5 and 10 Hz (FIG. 8). Interestingly, ranolazine caused only little use-dependent block of rNav1.8 channels (block of I_{Na} at 1, 5 and 10 Hz were 60.20±2.04%, 67.96±4.68% and 70.16±2.09% (p<0.05 when compared to 1 Hz), respectively). One possible explanation could be that dissociation of ranolazine from inactivated rNav1.8 channels is fast, much faster than its dissociation from inactivated hNav1.7 channels.

### Open Channel Block by Ranolazine

The voltage-dependent block (FIG. 3, Table 4, V_{0.5} holding potential experiments) and concentration-dependent shift in the mid-points (V_{1/2}) of inactivation of hNav1.7 and rNav1.8 (FIG. 5C, Table 7) caused by ranolazine, and the estimated (using Bean equation) K_{I} values of hNav1.7 and rNav1.8, suggest that ranolazine interacts with the inactivated state of these Na⁺ channels. However, it is unclear whether block of hNav1.7 or rNav1.8 Na⁺ channels by ranolazine with 10 msec depolarizing pulses at 1, 5 and 10 Hz also involved the transient open state in addition to the inactivated state of the channel. Wang and colleagues (Wang et al., 2008) have demonstrated that both muscle Nav1.4 and neuronal Nav1.7 are equally sensitive to ranolazine block, and they also demonstrated that the drug preferentially blocks the open state of these Na⁺ channels.

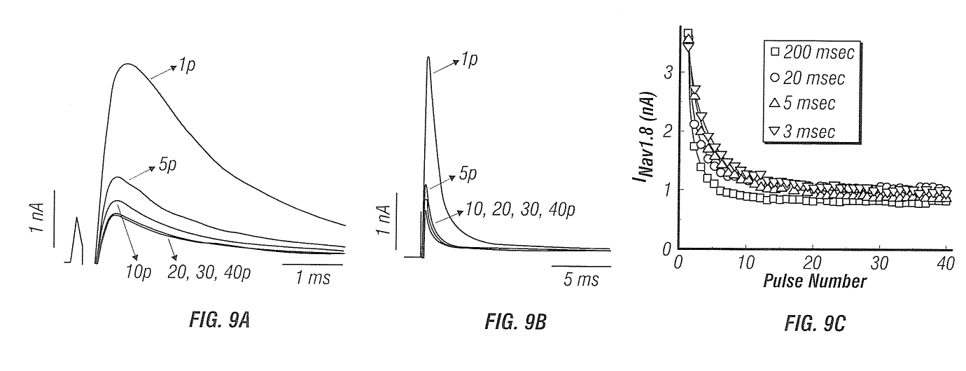

To investigate block of the open state of Nav1.7 and Nav1.8 channels, the effect of pulse duration on magnitude of use-dependent block by ranolazine was investigated. FIG. 9 shows representative records of rNav1.8 current elicited by 5 (FIG. 9A) or 200 msec (FIG. 9B) long test pulses to +50 mV at a frequency of 5 Hz in the presence of 100 μM ranolazine. Peak current elicited by each pulse was measured, normalized to the current of the first pulse, and plotted against the pulse number in FIG. 9C. The plot shows that the development of use-dependent block of rNav1.8 I_{Na} evoked by 3 (∇), 5 (Δ), 20 (∘) or 200 (□) msec-long test pulses to +50 mV in the presence of 100 μM ranolazine reached a steady-state of 71.69±0.85% (n=4-5 cells, each) with a time constant of 2.34±0.22 pulses.

In the absence of drug, repetitive pulses caused small reductions in Nav1.8 I_{Na} amplitude that increased with an increase of pulse duration from 3 to 5 to 20 to 200 msec by 16.89±4.59% (3 msec, n=5 cells) to 24.61±3.34% (5 msec, n=4 cells), 27.15±3.18% (20 msec, n=4 cells) and 30.43±2.55% (200 msec, n=4 cells).

The development of use-dependent block of hNav1.7 I_{Na} evoked by 2 (∇), 5 (Δ), 20 (∘) or 200 (□) msec-long test pulses to −20 mV was performed. In the presence of 100 μM ranolazine, use-dependent block of hNav1.7 reached a steady-state of 80.92±1.53% (n=5-6 cells) with a time constant of 5.83±0.19 pulses (data not shown). In the absence of drug, repetitive stimulation caused small or no reductions in the amplitude of I_{Na}. Thus, our data show that ranolazine blocked open states of hNav1.7 and rNav1.8 I_{Na}.

### Example 3

### Ranolazine-Treatment of CFA-Induced Hyperalgesia

The following Example demonstrates that ranolazine has a selective analgesic effect on mechanical allodynia and little if any effect on thermal hyperalgesia.

### Materials and Methods

All experiments were conducted in accordance with protocols that were approved and monitored by the LSU Medical Center Institutional Animal Care and Use Committee. Male Sprague Dawley rats (Harlan Sprague Dawley, Inc., Indianapolis, Ind.) weighing between 300-350 g were housed 1 animal to a cage and maintained at 25° C. and 60% humidity, on a 12 hour light/dark cycle and allowed access to food and water ad libitum. Rats were allowed to acclimate to their surroundings and for 1 hour/day to the testing apparatus for 1 week.

For determining baseline thresholds to thermal stimulation, groups of 9 rats were placed in Plexiglas chambers on a glass plate and were allowed free range of activity within the chamber. The glabrous surface of each hindpaw was stimulated sequentially through the glass plate using a halogen light source (Gould et al., 1997, 1998; Hargreaves et al., 1988). The latency of paw withdrawal from the onset of stimulation was measured using an IITC analgesiometer (IITC Life Science, Inc., Woodland Hills, Calif.). The stimulus was automatically discontinued after 10.7 seconds to avoid tissue damage. Each hindpaw was stimulated four times during each testing session.

Following thermal testing, thresholds to withdrawal from mechanical stimulation were recorded using an IITC Model 2290 electro-von Frey anesthsiometer (EVF; IITC Life Sciences, Inc., USA; Lewin et al., 1993, 1994; Gould et al., 2000b). For this, the rats were loosely restrained and allowed to accommodate to the restriction. The tip of the stimulating wand was then applied perpendicular to the skin at 4 sites on the dorsal surface of each hindpaw. The force applied to the paw at the time of paw withdrawal was recorded. The average force applied to each of the 4 sites was entered as the subject's response threshold for the interval and used in all further calculations. A ceiling of 250 g of force was imposed to prevent tissue injury from EVF testing.

Baseline pain thresholds for thermal and mechanical stimulation were determined at 2 time points prior to the subcutaneous injection of 0.1 ml of CFA (Mycobacterium tuberculosis, Sigma) suspended in oil:saline (1:1) emulsion (0.5 mg Mycobacterium/ml emulsion) into one hindpaw and an equivalent volume of sterile saline into the contralateral paw. Post-CFA withdrawal thresholds were recorded on each of the next 2 days. On the third day following CFA injection, withdrawal thresholds were recorded in groups of 9 rats that then received randomized and blinded doses of ranolazine (reconstituted in isotonic saline (0.9%) at pH 3.0) either by intraperitoneal (i.p.) injection (0, 10, 20, and 50 mg/kg) or by oral gavage (p.o.; 0, 20, 50, 100, and 200 mg/kg). In order to determine the optimum dosing range for producing analgesia, initial reference doses between 10 and 1000 mg/kg were administered by i.p. injection.

Withdrawal thresholds to thermal and mechanical stimulation were reassessed, 30 minutes after the i.p. administration of ranolazine and 1 hour after oral gavage. The behavioral data was subjected to a repeated measures, mixed design analysis of variance (ANOVA) for an internal comparison of the difference between the experimentally-manipulated and contralateral paws to determine statistical significance for changes in the withdrawal latencies.

### Results

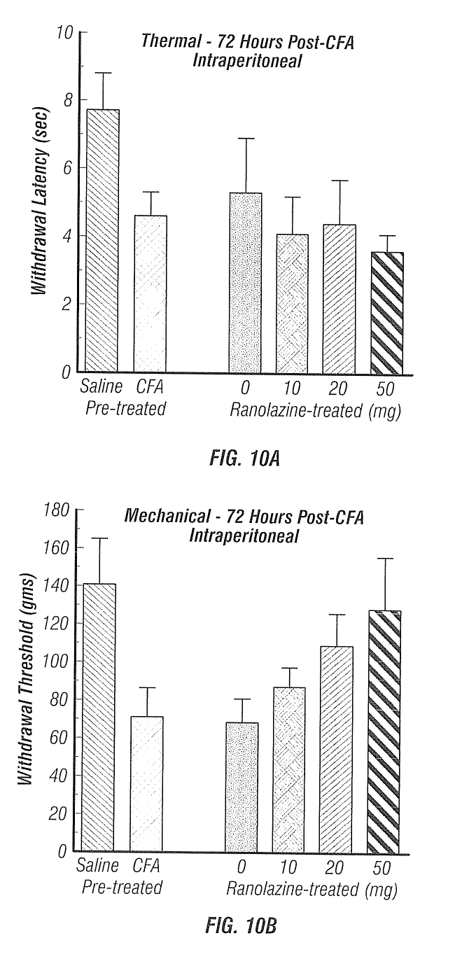

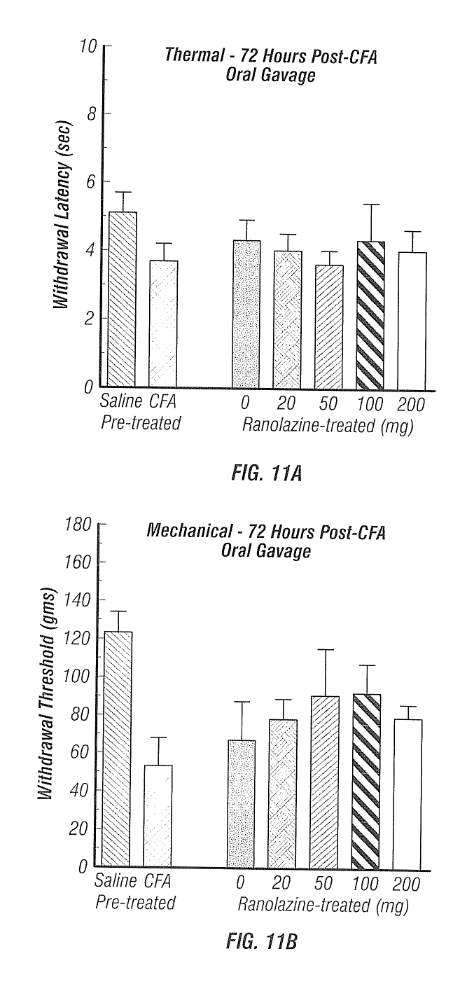

A single injection of CFA into the plantar surface of a rat hindpaw produces a profound and prolonged increase in sensitivity to both thermal and mechanical stimulation (Gould et al., 1997, 1998, 2004). The bars at the left of the graphs in FIGS. 10 and 11 depict the relative levels of thermal and mechanical stimulation necessary to produce paw withdrawal in 2 groups of rats 72 hours after the subcutaneous injection of CFA into one hindpaw when compared to the contralateral hindpaw that received an injection of an identical volume of normal saline. Stimulation of the CFA-injected hindpaw in vehicle-treated rats (0.9% isotonic saline; pH 3.0) revealed no significant difference in the response to either form of stimulation.

The addition of ranolazine clearly reduced paw sensitivity to mechanical stimulation in a dose-dependent fashion, but no significant effect on paw sensitivity was observed with thermal stimulation. Adverse effects were noted following i.p. administration only when doses at or above 100 mg/kg were given. The effects tended to be more pronounced at progressively higher doses. The adverse behavioral effects included bradykinesia, motor sluggishness manifested by slow response to stimulation and impaired performance on rotarod testing (Taylor et al., personal communication), muscle fasciculation and twitching, and convulsions. Death occurred in 50% of the rats treated with doses of 100 mg/kg.

A similar analgesic effect, specific for mechanical allodynia, was observed when ranolazine was administered by oral gavage (FIG. 11). Larger doses, however, were required to produce analgesia than when the drug was administered by i.p. injection. Unlike the i.p. route of administration, a plateau to the analgesic response was noted following oral gavage. The maximum response was achieved at a dose of 100 mg/kg. A non-significant trend toward a reduction in analgesic effect was observed at the 200 mg/kg dose.

Only at the highest p.o. dose did the ranolazine-treated rats develop an adverse event of respiratory strider approximately 1 hour after drug administration. The adverse pulmonary effect resolved within 24 hours of gavage and was not observed in vehicle-treated controls.

We claim:

1. A method of treatment or prevention of pain comprising the step of administering to a patient in need thereof a therapeutically effective amount, or a prophylactically effective amount, of Ranolazine, or a pharmaceutically acceptable salt thereof.

2. The method of claim 1, wherein the Ranolazine is administered for the treatment or prevention of neuropathic or nociceptive pain.

3. The method of claim 2, wherein Ranolazine is administered for the treatment or prevention of nociceptive pain.

4. The method of claim 3, wherein the nociceptive pain is mechanical, chemical, and/or inflammatory.

5. The method of claim 4, wherein the nociceptive pain is inflammatory.

6. The method of claim 2, wherein Ranolazine is administered for the treatment or prevention of neuropathic pain.

7. The method of claim 6, wherein the neuropathic pain is the result of a sodium channelopathy, polyneuropathy, autonomic neuropathy, mononeuropathy, or mononeuritis multiplex.

8. The method of claim 7, wherein the neuropathic pain is the result of a channelopathy.

9. The method of claim 8, wherein the pain is the result of erythromelalgia or paroxysmal extreme pain disorder.

10. The method of claim 1, wherein the Ranolazine is administered for the treatment or prevention pain resulting from, or associated with, traumatic nerve injury, nerve compression or entrapment, postherpetic neuralgia, trigeminal neuralgia, diabetic neuropathy, cancer and/or chemotherapy.

11. The method of claim 1, wherein the Ranolazine is administered for the treatment or prevention of chronic lower back pain.

12. The method of claim 1, wherein the Ranolazine is administered for the treatment or prevention of HIV- and HIV treatment-induced neuropathy, chronic pelvic pain, neuroma pain, complex regional pain syndrome, chronic arthritic pain and related neuralgias.

13. The method of claim 1, wherein the Ranolazine is administered as a local anesthesia.

14. A method for neuroprotection under ischaemic conditions caused by stroke or neural trauma comprising the step of administering to a patient in need thereof a therapeutically effective amount, or a prophylactically effective amount, of Ranolazine, or a pharmaceutically acceptable salt thereof.
